SEQUENCE LISTING (93 sequences)

SEQ ID NO: 1 - DNA, 23 bp, Artificial Sequence
  Description of Artificial Sequence Synthetic primer
gctctcatggaatggctaaagac

SEQ ID NO: 2 - DNA, 21 bp, Artificial Sequence
  Description of Artificial Sequence Synthetic probe
tcaccgtgcccagtgagcgag

SEQ ID NO: 3 - DNA, 23 bp, Artificial Sequence
  Description of Artificial Sequence Synthetic primer
gcattttggacaaagcgtctacg

SEQ ID NO: 4 - DNA, 29 bp, Artificial Sequence
  Description of Artificial Sequence Synthetic primer
gggattttgggatttgtgttcacgctcac

SEQ ID NO: 5 - DNA, 29 bp, Artificial Sequence
  Description of Artificial Sequence Synthetic probe
tacgctgcagtcctcgctcagtgggcacg

SEQ ID NO: 6 - DNA, 29 bp, Artificial Sequence
  Description of Artificial Sequence Synthetic primer
ttcccattaagggcattttggacaaagcg

SEQ ID NO: 7 - DNA, 37 bp, Artificial Sequence
  Description of Artificial Sequence Synthetic primer
taaagacaagaccaatcttgtcacctctgactaaggg

SEQ ID NO: 8 - DNA, 29 bp, Artificial Sequence
  Description of Artificial Sequence Synthetic primer
ttaccattgagggcattttggacaaagcg

SEQ ID NO: 9 - DNA, 24 bp, Artificial Sequence
  Description of Artificial Sequence Synthetic primer
ttacagtggaggatgaagaagatg

SEQ ID NO: 10 - DNA, 30 bp, Artificial Sequence
  Description of Artificial Sequence Synthetic probe
cattaagacgctcgaagagtgaattgagga

SEQ ID NO: 11 - DNA, 20 bp, Artificial Sequence
  Description of Artificial Sequence Synthetic primer
ctcgaattggctttgaatgt

SEQ ID NO: 12 - DNA, 25 bp, Artificial Sequence
  Description of Artificial Sequence Synthetic primer
tggatacaagtccttatcaactctg

SEQ ID NO: 13 - DNA, 23 bp, Artificial Sequence
  Description of Artificial Sequence Synthetic probe
tcgaagagtgagttgaggatccg

SEQ ID NO: 14 - DNA, 21 bp, Artificial Sequence
  Description of Artificial Sequence Synthetic primer
tgctcttgaccaaattgggat

SEQ ID NO: 15 - DNA, 23 bp, Artificial Sequence
  Description of Artificial Sequence Synthetic primer
gttgctaaacttgttgctactga

SEQ ID NO: 16 - DNA, 22 bp, Artificial Sequence
  Description of Artificial Sequence Synthetic probe
ttgaggatccgatggccatctt

SEQ ID NO: 17 - DNA, 18 bp, Artificial Sequence
  Description of Artificial Sequence Synthetic primer
gctgctcgaattggcttt

SEQ ID NO: 18 - DNA, 21 bp, Artificial Sequence
  Description of Artificial Sequence Synthetic primer
tggtgataatcggtgctcttg

SEQ ID NO: 19 - DNA, 23 bp, Artificial Sequence
  Description of Artificial Sequence Synthetic primer
gctaaacttgttgctactgatga

SEQ ID NO: 20 - DNA, 20 bp, Artificial Sequence
  Description of Artificial Sequence Synthetic primer
tcggtgctcttgaccaaatt

SEQ ID NO: 21 - DNA, 24 bp, Artificial Sequence
  Description of Artificial Sequence Synthetic primer
tacaagtccttatcaactctgcat

SEQ ID NO: 22 - DNA, 20 bp, Artificial Sequence
  Description of Artificial Sequence Synthetic primer
cttgttgctaaacttgttgc

SEQ ID NO: 23 - DNA, 23 bp, Artificial Sequence
  Description of Artificial Sequence Synthetic probe
tcggatcctcaactcactcttcg

SEQ ID NO: 24 - DNA, 16 bp, Artificial Sequence
  Description of Artificial Sequence Synthetic primer
tcagctgctcgaattg

SEQ ID NO: 25 - DNA, 23 bp, Artificial Sequence
  Description of Artificial Sequence Synthetic probe
tcggatcctcaattcactcttcg

SEQ ID NO: 26 - DNA, 18 bp, Artificial Sequence
  Description of Artificial Sequence Synthetic primer
tttcagctgctcgaattg

SEQ ID NO: 27 - DNA, 25 bp, Artificial Sequence
  Description of Artificial Sequence Synthetic primer
catcggatcctcaattcactcttcg

SEQ ID NO: 28 - DNA, 35 bp, Artificial Sequence
  Description of Artificial Sequence Synthetic probe
aatgaaggacattcaaagccaattcgagcagctga

SEQ ID NO: 29 - DNA, 25 bp, Artificial Sequence
  Description of Artificial Sequence Synthetic primer
cttgaccaaattgggataagactcc

SEQ ID NO: 30 - DNA, 29 bp, Artificial Sequence
  Description of Artificial Sequence Synthetic probe
caaagccaattcgagcagctgaaactgcg

SEQ ID NO: 31 - DNA, 25 bp, Artificial Sequence
  Description of Artificial Sequence Synthetic primer
gtgataatcggtgctcttgaccaaa

SEQ ID NO: 32 - DNA, 25 bp, Artificial Sequence
  Description of Artificial Sequence Synthetic primer
aacatgaccacaacacaaattgagg

SEQ ID NO: 33 - DNA, 35 bp, Artificial Sequence
  Description of Artificial Sequence Synthetic probe
tcctgcttcaaagtttatagtggcattggttgctc

SEQ ID NO: 34 - DNA, 25 bp, Artificial Sequence
  Description of Artificial Sequence Synthetic primer
gtaatcaagggctctttgccatgaa

SEQ ID NO: 35 - DNA, 23 bp, Artificial Sequence
  Description of Artificial Sequence Synthetic primer
tcacaacacaaattgaggtgggt

SEQ ID NO: 36 - DNA, 20 bp, Artificial Sequence
  Description of Artificial Sequence Synthetic primer
ttggccagggtagtcaaggg

SEQ ID NO: 37 - DNA, 22 bp, Artificial Sequence
  Description of Artificial Sequence Synthetic primer
ctgtttaggcggttttgaccag

SEQ ID NO: 38 - DNA, 39 bp, Artificial Sequence
  Description of Artificial Sequence Synthetic primer
gttgctaaacttgttgctactgatgatcttacagtggag

SEQ ID NO: 39 - DNA, 35 bp, Artificial Sequence
  Description of Artificial Sequence Synthetic probe
agacgctcgaagagtgagttgaggatccgatggcc

SEQ ID NO: 40 - DNA, 31 bp, Artificial Sequence
  Description of Artificial Sequence Synthetic primer
gctgctcgaattggttttgaatgtccttcat

SEQ ID NO: 41 - DNA, 39 bp, Artificial Sequence; identical to SEQ ID NO: 38
  Description of Artificial Sequence Synthetic primer

SEQ ID NO: 42 - DNA, 35 bp, Artificial Sequence; identical to SEQ ID NO: 39
  Description of Artificial Sequence Synthetic probe

SEQ ID NO: 43 - DNA, 31 bp, Artificial Sequence; identical to SEQ ID NO: 40
  Description of Artificial Sequence Synthetic primer

SEQ ID NO: 44 - DNA, 25 bp, Artificial Sequence
  Description of Artificial Sequence Synthetic primer
ccaccaacatcaaagaaggatcaaa

SEQ ID NO: 45 - DNA, 28 bp, Artificial Sequence
  Description of Artificial Sequence Synthetic probe
tgatcctgcattgtcacagtaccatcct

SEQ ID NO: 46 - DNA, 22 bp, Artificial Sequence
  Description of Artificial Sequence Synthetic primer
ctttacaagtgtcagcctgtgg

SEQ ID NO: 47 - DNA, 25 bp, Artificial Sequence
  Description of Artificial Sequence Synthetic primer
tcccctctatgtacaaccaacacaa

SEQ ID NO: 48 - DNA, 28 bp, Artificial Sequence
  Description of Artificial Sequence Synthetic probe
tgatcctgcattatcacaataccatcct

SEQ ID NO: 49 - DNA, 24 bp, Artificial Sequence
  Description of Artificial Sequence Synthetic primer
ctttacatgtttcagcttgtggga

SEQ ID NO: 50 - DNA, 39 bp, Artificial Sequence
  Description of Artificial Sequence Synthetic primer
cctcttgtcacaataatatgtacatataggcatgcacct

SEQ ID NO: 51 - DNA, 35 bp, Artificial Sequence
  Description of Artificial Sequence Synthetic probe
tgacaccacccttcgataccacccatgtgatatct

SEQ ID NO: 52 - DNA, 29 bp, Artificial Sequence
  Description of Artificial Sequence Synthetic primer
gtgatatagcttctatggtccacagtttt

SEQ ID NO: 53 - DNA, 35 bp, Artificial Sequence
  Description of Artificial Sequence Synthetic probe
acaccagccctcaataccacccatatggtatctgt

SEQ ID NO: 54 - DNA, 40 bp, Artificial Sequence
  Description of Artificial Sequence Synthetic primer
ttagatctaatagtgatatagcttctatggtccatagttt

SEQ ID NO: 55 - DNA, 40 bp, Artificial Sequence
  Description of Artificial Sequence Synthetic primer
tcagatctaataatgatatggcttcaatggtccacagttt

SEQ ID NO: 56 - DNA, 20 bp, Artificial Sequence
  Description of Artificial Sequence Synthetic primer
gtttccgtcttgctcgtatc

SEQ ID NO: 57 - DNA, 24 bp, Artificial Sequence
  Description of Artificial Sequence Synthetic probe
cgcaagttcttcagcgaaaagcac

SEQ ID NO: 58 - DNA, 21 bp, Artificial Sequence
  Description of Artificial Sequence Synthetic primer
tttcacctccagtatggaacc

SEQ ID NO: 59 - DNA, 19 bp, Artificial Sequence
  Description of Artificial Sequence Synthetic primer
caatgcaacgttctccaac

SEQ ID NO: 60 - DNA, 20 bp, Artificial Sequence
  Description of Artificial Sequence Synthetic probe
tgcaggatgcagcgccttac

SEQ ID NO: 61 - DNA, 19 bp, Artificial Sequence
  Description of Artificial Sequence Synthetic primer
taacggttgcttgttcagc

SEQ ID NO: 62 - DNA, 23 bp, Artificial Sequence
  Description of Artificial Sequence Synthetic primer
aatcttcgtaaaacgttcgtgtc

SEQ ID NO: 63 - DNA, 28 bp, Artificial Sequence
  Description of Artificial Sequence Synthetic probe
cacttttaccgtggtgtcgatgtcaaac

SEQ ID NO: 64 - DNA, 20 bp, Artificial Sequence
  Description of Artificial Sequence Synthetic primer
cgaagagattgtcaacaggt

SEQ ID NO: 65 - DNA, 17 bp, Artificial Sequence
  Description of Artificial Sequence Synthetic primer
gtccgagaccaatgtgc

SEQ ID NO: 66 - DNA, 29 bp, Artificial Sequence
  Description of Artificial Sequence Synthetic probe
ccgttccctacaacgagcctaaattcata

SEQ ID NO: 67 - DNA, 18 bp, Artificial Sequence
  Description of Artificial Sequence Synthetic primer
caggcagcccgatctatt

SEQ ID NO: 68 - DNA, 23 bp, Artificial Sequence
  Description of Artificial Sequence Synthetic primer
atcttcgtaaaacgttcgtgtcc

SEQ ID NO: 69 - DNA, 29 bp, Artificial Sequence
  Description of Artificial Sequence Synthetic probe
tttgacatcgacaccacggtaaaagtgcg

SEQ ID NO: 70 - DNA, 22 bp, Artificial Sequence
  Description of Artificial Sequence Synthetic primer
gcgaagagattgtcaacaggtt

SEQ ID NO: 71 - DNA, 25 bp, Artificial Sequence
  Description of Artificial Sequence Synthetic primer
gtgtcaaacaaaggagtagatactg

SEQ ID NO: 72 - DNA, 35 bp, Artificial Sequence
  Description of Artificial Sequence Synthetic probe
ttccttccagcttgttgacatagtataaagtgttg

SEQ ID NO: 73 - DNA, 25 bp, Artificial Sequence
  Description of Artificial Sequence Synthetic primer
taggttccccttttacataaaggtt

SEQ ID NO: 74 - DNA, 25 bp, Artificial Sequence
  Description of Artificial Sequence Synthetic primer
taggagctatagtgtcatgttatgg

SEQ ID NO: 75 - DNA, 35 bp, Artificial Sequence
  Description of Artificial Sequence Synthetic probe
tgacacataatcacaaccattagaaaatgtcttta

SEQ ID NO: 76 - DNA, 25 bp, Artificial Sequence
  Description of Artificial Sequence Synthetic primer
tccttccagcttgtttacatagtat

SEQ ID NO: 77 - DNA, 25 bp, Artificial Sequence
  Description of Artificial Sequence Synthetic primer
tggttgtgattatgtgtcaaacaaa

SEQ ID NO: 78 - DNA, 35 bp, Artificial Sequence
  Description of Artificial Sequence Synthetic probe
tagatactgtgtcagtgggcaacactttatactat

SEQ ID NO: 79 - DNA, 15 bp, Artificial Sequence
  Description of Artificial Sequence Synthetic primer
gccttccagcttgtt

SEQ ID NO: 80 - DNA, 25 bp, Artificial Sequence
  Description of Artificial Sequence Synthetic primer
tctaatggttgtgattatgtgtcaa

SEQ ID NO: 81 - DNA, 31 bp, Artificial Sequence
  Description of Artificial Sequence Synthetic probe
caaaggagtagatactgtatcagtgggcaac

SEQ ID NO: 82 - DNA, 25 bp, Artificial Sequence
  Description of Artificial Sequence Synthetic primer
gccttccagcttgttgacatagtat

SEQ ID NO: 83 - DNA, 18 bp, Artificial Sequence
  Description of Artificial Sequence Synthetic primer
tttgccttccagcttgtt

SEQ ID NO: 84 - DNA, 21 bp, Artificial Sequence
  Description of Artificial Sequence Synthetic probe
tcctgcattatcacaatacca

SEQ ID NO: 85 - DNA, 25 bp, Artificial Sequence
  Description of Artificial Sequence Synthetic probe
atcctgcattatcacaataccatcc

SEQ ID NO: 86 - DNA, 21 bp, Artificial Sequence
  Description of Artificial Sequence Synthetic probe
tcctgcattatcacagtacca

SEQ ID NO: 87 - DNA, 25 bp, Artificial Sequence
  Description of Artificial Sequence Synthetic probe
taccatcctctatcagtccttgtta

SEQ ID NO: 88 - DNA, 25 bp, Artificial Sequence
  Description of Artificial Sequence Synthetic primer
tctaatggttgtgattatgtgtcaa

SEQ ID NO: 89 - DNA, 31 bp, Artificial Sequence; identical to SEQ ID NO: 81
  Description of Artificial Sequence Synthetic probe

SEQ ID NO: 90 - DNA, 25 bp, Artificial Sequence
  Description of Artificial Sequence Synthetic primer
gccttccagcttgttgacatagtat

SEQ ID NO: 91 - DNA, 10 bp, Artificial Sequence
  Description of Artificial Sequence Synthetic probe
aaacacgtgc

SEQ ID NO: 92 - DNA, 10 bp, Artificial Sequence
  Description of Artificial Sequence Synthetic probe
ccttgttcca

SEQ ID NO: 93 - DNA, 10 bp, Artificial Sequence
  Description of Artificial Sequence Synthetic probe
cagggacgat

(19) United States
(12) Patent Application Publication
(10) Pub. No.: US 2014/0127674 A1
(43) Pub. Date: May 8, 2014

(54) OPTIMIZED PROBES AND PRIMERS AND METHODS OF USING SAME FOR THE BINDING, DETECTION, DIFFERENTIATION, ISOLATION AND SEQUENCING OF INFLUENZA A; INFLUENZA B AND RESPIRATORY SYNCYTIAL VIRUS

(71) Applicant: Intelligent Medical Devices, Inc., Cambridge, MA (US)

(72) Inventors: Sean Bradley, Newton, MA (US); Paul Labrousse, Hubbardston, MA (US); Chesley Leslin, Boston, MA (US); Damien Slater, Roslindale, MA (US); James R. Hully, Mundelein, IL (US); Alice A. Jacobs, Cambridge, MA (US); Heather Kiefer, Westford, MA (US); Juan Anzola, Bogota (CO)

(73) Assignee: Intelligent Medical Devices, Inc., Cambridge, MA (US)

(21) Appl. No.: 14/063,462

(22) Filed: Oct. 25, 2013

Related U.S. Application Data
(60) Provisional application No. 61/718,508, filed on Oct. 25, 2012.

(51) Int. Cl. C12Q 1/70

(52) U.S. Cl. CPC C12Q 1/701
USPC 435/5; 536/24.32

(57) ABSTRACT

Described herein are primers and probes useful for the binding, detecting, differentiating, isolating, and sequencing of influenza A, influenza B and RSV viruses.

### RELATED APPLICATION

This application claims the benefit of U.S. Provisional Application No. 61/718,508, filed on Oct. 25, 2012, the contents of which are incorporated by reference herein in their entirety.

### BACKGROUND

Influenza viruses are enveloped, single stranded negative-sense, segmented genome RNA viruses of the family Orthomyxoviridae. Influenza viruses are divided into three distinct types A, B and C; only types A and B have been identified as a concern in human pathogenicity.

Influenza A viruses are subtyped based upon antigenicity and genetics of their surface proteins, hemaglutinin (HA) and neuraminidase (NA), which are the major targets of the host organism's immune system. Contemporary circulating seasonal influenza A viruses are classified as H1N1 or H3N2. Influenza B viruses are mainly found in humans. All types of influenza have been shown to undergo antigenic shift and drift, though at different rates.

Seasonal influenza strains (such as influenza A and influenza B) customarily peak in incidence and disease with a seasonal periodicity.

In the United States, more than 200,000 people are hospitalized from influenza-related causes and an average of 36,000 people die from influenza-related complications annually. Transmission of the influenza virus occurs by aerosol, such as coughing and sneezing, and with contact with nasal discharge. Close contact and indoor environments favor transmission. Humans infected with seasonal influenza virus shed virus and may be able to infect others from 1 day before showing signs of illness to 5 to 7 days after becoming ill. The human influenza viruses are easily transmitted from human to human.

Symptoms of influenza A and B infections are characterized by fever, chills, anorexia, headache, myalgia, weakness, sneezing, rhinitis, sore throat and a nonproductive cough. In approximately half of all cases, nausea and vomiting may occur.

Traditional testing for influenza is performed using viral culture methods. Currently, the majority of influenza testing is performed using rapid lateral flow assays or rapid antigen detection assays, which are designed to either detect and discriminate influenza A and influenza B, or simply detect influenza A.

Respiratory syncytial virus (RSV) is an enveloped, single stranded negative sense, non-segmented genome RNA virus of the family Paramyxoviridae. RSV is a major cause of bronchiolitis and pneumonia in infants under the age of one and infects almost all children by the age of three. RSV infection of adults, especially among the elderly and immunocompromised individuals, has increased significantly in recent years. Moreover, RSV infections may trigger or exacerbate respiratory conditions, including asthma.

Complications associated with respiratory syncytial virus include inflammation of the lungs (pneumonia) or the lung's airways (bronchiolitis). RSV can also be found to infect the middle ear of infants and young children. Once infected, recurrences of RSV infection are fairly common and pose serious health risks for elderly and immunocompromised individuals.

The RSV genome contains 10 genes, which are transcribed by a virally encoded RNA polymerase. The polymerase complex contains the polymerase L protein, phosphoprotein P and transcription elongation factor M1-2 protein. The major RSV antigens are an attachment glycoprotein (G) and a fusion glycoprotein (F). A nucleocapsid protein (N) is an essential structural protein.

Influenza detection and differentiation, in combination with RSV detection, would allow for improved treatments of viral infections. A rapid and accurate diagnostic test panel for the simultaneous detection and differentiation of influenza A, influenza B, and RSV virus, therefore, would provide clinicians with an effective tool for identifying patients symptomatic for one or more of the respiratory viruses and subsequently supporting effective treatment regimens.

### SUMMARY

The present disclosure provides compositions and assays for detecting the presence of influenza and respiratory syncytial viruses (influenza A, influenza B and RSV).

Described herein are nucleic acid probes and primers for binding, detecting, discriminating, isolating and sequencing all or the majority of known, characterized variants of influenza A, influenza B and respiratory syncytial viruses (RSV), with a high degree of sensitivity and specificity. The above described assay can also include a process control.

When used alone, each individual primer/probe set or a probe alone can specifically detect all or most known variants of the corresponding virus type (i.e., influenza A, influenza B or RSV) without cross-reacting with the other two virus types. In combination, moreover, the primer/probe sets or probe sets can simultaneously detect two or more of such virus types. Accordingly, in one embodiment, the present disclosure provides individual primer/probe sequences, primer/probe sets, and groups of primer/probe sets, for carrying out such detections.

A diagnostic test or tests that distinguish influenza A, influenza B and respiratory syncytial viruses simultaneously in humans are important because such detection is critical in early patient identification and treatment. The assays described herein also aid in the intervention of the spread of these highly infectious viruses.

The assays described herein are used to identify or confirm the identification of influenza A, influenza B and respiratory syncytial viruses. The assays can be performed in a single testing scheme consisting of simultaneous analysis of the same patient sample in one reaction. The reaction can be directed to, for example, the identification of influenza A, influenza B and respiratory syncytial virus.

Alternatively, the assays may be performed in a single testing scheme consisting of simultaneous analysis of the same patient sample in two separate reactions. The first reaction may consist of, for example, the identification of influenza A and influenza B. The second reaction may consist of, for example, the identification of respiratory syncytial virus. Assay results for all tests can be obtained and/or delivered simultaneously.

Many facilities utilize viral culture-based methods for the determination and detection of respiratory infections, which requires days to obtain the results. The methods of detection of the present invention described herein can be carried out within a minimal number of hours, allowing clinicians to rapidly determine the appropriate treatment options for individuals infected with respiratory virus(es).

One embodiment is directed to an isolated nucleic acid sequence comprising a sequence selected from the group consisting of: SEQ ID NOS: 1-90.

One embodiment is directed to a method of hybridizing one or more isolated nucleic acid sequences comprising a sequence selected from the group consisting of: SEQ ID NOS: 1-55, 71-90 to an influenza A, influenza B and/or RSV sequence, comprising contacting one or more isolated nucleic acid sequences to a sample comprising the influenza and/or RSV sequence under conditions suitable for hybridization. In a particular embodiment, the sequence is a genomic sequence, a naturally occurring plasmid, a naturally occurring transposable element, a template sequence or a sequence derived from an artificial construct. In a particular embodiment, the method(s) further comprise isolating and/or sequencing the hybridized influenza and/or RSV sequence.

One embodiment is directed to a primer set comprising at least one forward primer selected from the group consisting of SEQ ID NOS: 1, 4, 7, 9, 12, 15, 19, 21, 22, 27, 32, 35, 38, 41, 44, 47, 50, 56, 59, 62, 65, 68, 71, 74, 77, 80 and 88; and at least one reverse primer selected from the group consisting of SEQ ID NOS: 3, 6, 8, 11, 14, 17, 18, 20, 24, 26, 29, 31, 34, 36, 37, 40, 43, 46, 49, 52, 54, 55, 58, 61, 64, 67, 70, 73, 76, 79, 82, 83 and 90.

One embodiment is directed to a primer set (at least one forward primer and at least one reverse primer) selected from the group consisting of: Groups 1-38 of Table 3.

One embodiment is directed to a method of producing a nucleic acid product, comprising contacting one or more isolated nucleic acid sequences selected from the group consisting of SEQ ID NOS: 1, 3, 4, 6, 7, 8, 9, 11, 12, 14, 15, 17, 18, 19, 20, 21, 22, 24, 26, 27, 29, 31, 32, 34, 35, 36, 37, 38, 40, 41, 43, 44, 46, 47, 49, 50, 52, 54, 55, 56, 58, 59, 61, 62, 64, 65, 67, 68, 70, 71, 73, 74, 76, 77, 79, 80, 82, 83, 88 and 90 to a sample comprising an influenza and/or RSV sequence under conditions suitable for nucleic acid polymerization. In a particular embodiment, the nucleic acid product is an influenza and/or RSV amplicon produced using at least one forward primer selected from the group consisting of SEQ ID NOS: 1, 4, 7, 9, 12, 15, 19, 21, 22, 27, 32, 35, 38, 41, 44, 47, 50, 56, 59, 62, 65, 68, 71, 74, 77, 80 and 88, and at least one reverse primer selected from the group consisting of SEQ ID NOS: 3, 6, 8, 11, 14, 17, 18, 20, 24, 26, 29, 31, 34, 36, 37, 40, 43, 46, 49, 52, 54, 55, 58, 61, 64, 67, 70, 73, 76, 79, 82, 83 and 90.

One embodiment is directed to a probe that hybridizes to an amplicon produced as described herein, e.g., using the primers described herein. In a particular embodiment, the probe comprises a sequence selected from the group consisting of SEQ ID NOS: 2, 5, 10, 13, 16, 23, 25, 28, 30, 33, 39, 42, 45, 48, 51, 53, 57, 60, 63, 66, 69, 72, 75, 78, 81, 84, 85, 86, 87 and 89. In a particular embodiment, the probe(s) is labeled with a detectable label selected from the group consisting of: a fluorescent label, a chemiluminescent label, a quencher, a radioactive label, biotin and gold.

One embodiment is directed to a set of probes that hybridize to an amplicon produced as described herein, e.g., using the primers described herein. In a particular embodiment, a first probe can comprise an influenza A sequence, for example, selected from the group consisting of SEQ ID NOS: 2 and 5; a second probe can comprise an influenza B sequence, for example, selected from the group consisting of SEQ ID NOS: 10, 13, 16, 23, 25, 28, 30, 33, 39 and 42 and a third probe can comprise an RSV sequence, for example, SEQ ID NOS: 45, 48, 51, 53, 72, 75, 78, 81, 84, 85, 86, 87 and 89.

One embodiment is directed to a set of probes that hybridize to an amplicon produced as described herein, e.g., using the primers described herein. In a particular embodiment, a first probe can comprise an influenza A sequence, for example, selected from the group consisting of SEQ ID NOS: 2 and 5; a second probe can comprise an influenza B sequence, for example, selected from the group consisting of SEQ ID NOS: 10, 13, 16, 23, 25, 28, 30, 33, 39 and 42; a third probe can comprise an RSV sequence, for example, SEQ ID NOS: 45, 48, 51, 53, 72, 75, 78, 81, 84, 85, 86, 87 and 89; and a fourth probe can comprise a process control sequence, for example, selected from the group consisting of SEQ ID NOS: 57, 60, 63, 66 and 69. In a particular embodiment, each of the probes is labeled with a different detectable label. In additional embodiments, one or more of the probes is labeled with the same detectable label.

One embodiment is directed to a probe that hybridizes directly to the genomic sequences of the target without amplification. In a particular embodiment, the probe comprises a sequence, for example, selected from the group consisting of SEQ ID NOS: 2, 5, 10, 13, 16, 23, 25, 28, 30, 33, 39, 42, 45, 48, 51, 53, 72, 75, 78, 81, 84, 85, 86, 87 and 89. In a particular embodiment, the probe(s) is labeled with a detectable label, for example, selected from the group consisting of: a fluorescent label, a chemiluminescent label, a quencher, a radioactive label, biotin and gold.

One embodiment, using any of the probe combinations described herein, is directed to a set of probes that hybridize directly to the genomic sequences of the target without amplification.

In one embodiment, the probe(s) is fluorescently labeled and the step of detecting the binding of the probe to the amplified product comprises measuring the fluorescence of the sample. In one embodiment, the probe comprises a fluorescent reporter moiety and a quencher of fluorescence-quenching moiety. Upon probe hybridization with the amplified product, the exonuclease activity of a DNA polymerase dissociates the probe's fluorescent reporter and the quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. In another embodiment, each probe in the multiplex reaction is labeled with a different distinguishable and detectable label.

In a particular embodiment, the probes are molecular beacons. Molecular beacons are single-stranded probes that form a stem-and-loop structure. A fluorophore is covalently linked to one end of the stem and a quencher is covalently linked to the other end of the stem forming a stem hybrid; fluorescence is quenched when the formation of the stem loop positions the fluorophore proximal to the quencher. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein may be designed and utilized as molecular beacons.

One embodiment is directed to a method for detecting influenza A, influenza B and/or RSV DNA in a sample, comprising: (a) contacting the sample with at least one forward primer comprising a sequence selected from the group consisting of: SEQ ID NOS: 1, 4 and 7 (influenza A); 9, 12, 15, 19, 21, 22, 27, 32, 35, 38 and 41 (influenza B); and 44, 47, 50, 71, 74, 77, 80 and 88 (RSV), and at least one reverse primer comprising a sequence selected from the group consisting of: SEQ ID NOS: 3, 6 and 8 (influenza A); 11, 14, 17, 18, 20, 24, 26, 29, 31, 34, 36, 37, 40 and 43 (influenza B); and 46, 49, 52, 54, 55, 73, 76, 79, 82, 83 and 90 (RSV); under conditions such that nucleic acid amplification occurs to yield an amplicon; and (b) contacting the amplicon with one or more probes comprising one or more sequences selected from the group consisting of: SEQ ID NOS: 2, 5 (influenza A); 10, 13, 16, 23, 25, 28, 30, 33, 39 and 42 (influenza B); and 45, 48, 51, 53, 72, 75, 78, 81, 84, 85, 86, 87 and 89 (RSV) under conditions such that hybridization of the probe to the amplicon occurs, wherein hybridization of the probe is indicative of influenza A and/or influenza B and/or RSV DNA in the sample.

The term “viral DNA” or “DNA of a virus” as used herein, when referring to an RNA virus, means a DNA that includes a nucleotide sequence complementary to a nucleotide sequence within the RNA virus. Generation of such DNA can be natural, such as with retroviruses that produce DNA intermediates. The DNA can also be prepared under lab conditions such as by reverse transcription.

In one embodiment, step (a) comprises contacting the sample with (i) at least one forward primer comprising a sequence selected from the group consisting of: SEQ ID NOS: 1, 4 and 7 (influenza A); (ii) at least one forward primer comprising a sequence selected from the group consisting of: SEQ ID NOS: 9, 12, 15, 19, 21, 22, 27, 32, 35, 38 and 41 (influenza B); and (iii) at least one forward primer comprising a sequence selected from the group consisting of: SEQ ID NOS: 44, 47, 50, 71, 74, 77, 80 and 88 (RSV), and (iv) at least one reverse primer comprising a sequence selected from the group consisting of: SEQ ID NOS: 3, 6 and 8 (influenza A); (v) at least one reverse primer comprising a sequence selected from the group consisting of: SEQ ID NOS: 11, 14, 17, 18, 20, 24, 26, 29, 31, 34, 36, 37, 40 and 43 (influenza B); and (iv) at least one reverse primer comprising a sequence selected from the group consisting of: SEQ ID NOS: 46, 49, 52, 54, 55, 73, 76, 79, 82, 83 and 90 (RSV); under conditions such that nucleic acid amplification occurs to yield an amplicon. In an alternative embodiment, the sample is contacted with any two of (i)-(iii) and any two of (iv)-(vi).

In a particular embodiment, each of the one or more probes is labeled with a different detectable label. In a particular embodiment, the one or more probes are labeled with the same detectable label. In a particular embodiment, the sample is selected from the group consisting of: saliva, fluids collected from the ear, eye, mouth, and respiratory airways, sputum, tears, or opharyngeal swabs, nasopharyngeal swabs, nasal swabs, throat swabs, nasopharyngeal aspirates, bronchoalveolar lavage fluid, skin swabs, nasal aspirates, nasal wash, and fluids and cells obtained by the perfusion of tissues of both human and animal origin. In one embodiment, the sample is from a human, is non-human in origin, or is derived from an inanimate object or environmental surfaces. In a particular embodiment, the at least one forward primer, the at least one reverse primer and the one or more probes are selected from the group consisting of: Groups 1-38 of Table 3. In a particular embodiment, the method(s) further comprise isolating and/or sequencing the influenza A, influenza B and/or RSV DNA.

One embodiment is directed to a primer set or collection of primer sets for amplifying DNA of an influenza A strain, comprising a nucleotide sequence selected from the group consisting of: (1) SEQ ID NOS: 1 and 3; and (2) SEQ ID NOS: 4, 6, 7 and 8.

One embodiment is directed to a primer set or collection of primer sets for amplifying DNA of an influenza B strain, comprising a nucleotide sequence selected from the group consisting of: (1) SEQ ID NO: 9 and 11; (2) SEQ ID NOS: 12, 14, 15 and 17; (3) SEQ ID NOS: 12, 17, 18, 19; (4) SEQ ID NOS: 12, 14, 17 and 19; (5) SEQ ID NOS: 12, 15, 17, 18; (6) SEQ ID NOS: 12, 15, 17, 20; (7) SEQ ID NOS: 15, 17, 18, 21; (8) SEQ ID NOS: 22, 24 and 26; (9) SEQ ID NOS: 12, 15 and 17; (10) SEQ ID NOS: 27 and 29; (11) SEQ ID NOS: 27 and 31: (12) SEQ ID NOS: 32, 34, 35 and 36; (13) SEQ ID NOS: 32, 34, 35 and 37; (14) SEQ ID NOS: 38 and 40 and (15) SEQ ID NOS: 41 and 43.

One embodiment is directed to a primer set or collection of primer sets for amplifying DNA of an RSV strain, comprising a nucleotide sequence selected from the group consisting of: (1) SEQ ID NOS: 44 and 46; (2) SEQ ID NOS: 47 and 49; (3) SEQ ID NOS: 50, 52, 54 and 55; (4) SEQ ID NOS: 71 and 73; (5) SEQ ID NOS: 74 and 76; (6) SEQ ID NOS: 77 and 79; (7) SEQ ID NOS: 76 and 80; (8) SEQ ID NOS: 80 and 82; (9) SEQ ID NOS: 77 and 83 and (10) SEQ ID NOS: 88 and 90.

One embodiment is directed to the simultaneous detection and differentiation in a multiplex format of (1) influenza A, and/or (2) influenza B, and/or (3) RSV.

One embodiment is directed to a primer set or collection of primer sets for amplifying DNA of influenza A, and/or influenza B and/or RSV simultaneously, comprising:

(a) a primer set selected from the group consisting of (1) SEQ ID NOS: 1 and 3; and (2) SEQ ID NOS: 4, 6, 7 and 8 (forward and reverse primers for amplifying DNA of influenza A); and

(b) a primer set selected from the group consisting of (1) SEQ ID NO: 9 and 11; (2) SEQ ID NOS: 12, 14, 15 and 17; (3) SEQ ID NOS: 12, 17, 18, 19; (4) SEQ ID NOS: 12, 14, 17 and 19; (5) SEQ ID NOS: 12, 15, 17, 18; (6) SEQ ID NOS: 12, 15, 17, 20; (7) SEQ ID NOS: 15, 17, 18, 21; (8) SEQ ID NOS: 22, 24 and 26; (9) SEQ ID NOS: 12, 15 and 17; (10) SEQ ID NOS: 27 and 29; (11) SEQ ID NOS: 27 and 31: (12) SEQ ID NOS: 32, 34, 35 and 36; (13) SEQ ID NOS: 32, 34, 35 and 37; (14) SEQ ID NOS: 38 and 40 and (15) SEQ ID NOS: 41 and 43 (forward and reverse primers for amplifying DNA of influenza B); and

(c) a primer set selected from the group consisting of (1) SEQ ID NOS: 44 and 46; (2) SEQ ID NOS: 47 and 49, (3) SEQ ID NOS: 50, 52, 54 and 55; (4) SEQ ID NOS: 71 and 73; (5) SEQ ID NOS: 74 and 76; (6) SEQ ID NOS: 77 and 79; (7) SEQ ID NOS: 76 and 80; (8) SEQ ID NOS: 80 and 82; (9) SEQ ID NOS: 77 and 83; and (10) SEQ ID NOS: 88 and 90 (forward and reverse primers for amplifying DNA of RSV). In one embodiment, the collection of primer sets comprises (a) and (b), or alternatively (a) and (c), or alternatively (b) and (c).

Another embodiment provides a collection of primer sets comprising at least two, or alternatively at least three, or all four of the following:

(a) a primer set selected from Groups 1-2 of Table 3,

(b) a primer set selected from Groups 3-19 of Table 3,

(c) a primer set selected from Groups 20-22, 28-38 of Table 3, and

(d) a primer set selected from Groups 23-27 of Table 3. In one embodiment, the collection of primer sets comprises (a) and (b); or (a) and (c); or (b) and (c); or (a), (b) and (c); or (a) (b) and (d); or (a), (c) and (d); or (b), (c) and (d). In another embodiment, the collection of primer set further includes a probe sequence in the corresponding Group of Table 3.

A particular embodiment is directed to oligonucleotide probes for binding to DNA of influenza A, and/or influenza B and/or RSV, comprising nucleotide sequence(s) selected from the group consisting of SEQ ID NOS: 2, 5 (influenza A probes); 10, 13, 16, 23, 25, 28, 30, 33, 39 and 42 (influenza B probes); and 45, 48, 51, 53, 72, 75, 78, 81, 84, 85, 86, 87 and 89 (RSV probes).

One embodiment is directed to a kit for detecting DNA of an influenza and/or RSV virus in a sample, comprising one or more probes comprising a sequence selected from the group consisting of: SEQ ID NOS: 2, 5 (influenza A probes); 10, 13, 16, 23, 25, 28, 30, 33, 39 and 42 (influenza B probes); and 45, 48, 51, 53, 72, 75, 78, 81, 84, 85, 86, 87 and 89 (RSV probes). In a particular embodiment, the kit further comprises one or more probes comprising a sequence selected from the group consisting of: SEQ ID NOS: 57, 60, 63, 66 and 69 (Process Control probes). In a particular embodiment, the kit further comprises a) at least one forward primer comprising the sequence selected from the group consisting of: SEQ ID NOS: 1, 4 and 7 (influenza A); 9, 12, 15, 19, 21, 22, 27, 32, 35, 38 and 41 (influenza B); and 44, 47, 50, 71, 74, 77, 80 and 88 (RSV), and at least one reverse primer comprising a sequence selected from the group consisting of: SEQ ID NO: 3, 6 and 8 (influenza A); 11, 14, 17, 18, 20, 24, 26, 29, 31, 34, 36, 37, 40 and 43 (influenza B); and 46, 49, 52, 54, 55, 73, 76, 79, 82, 83 and 90 (RSV). In a particular embodiment, the kit further comprises (a) at least one forward primer comprising the sequence selected from the group consisting of: SEQ ID NOS: 56, 59, 62, 65 and 68 (Process Control); and (b) at least one reverse primer comprising the sequence selected from the group consisting of: SEQ ID NOS: 58, 61, 64, 67 and 70 (Process Control). In a particular embodiment, the kit further comprises reagents for isolating and/or sequencing the DNA in the sample. In a particular embodiment, the one or more probes are labeled with different detectable labels. In a particular embodiment, the one or more probes are labeled with the same detectable labels. In a particular embodiment, the at least one forward primer, the at least one reverse primer and the one or more probes are selected from the group consisting of: Groups 1-38 of Table 3.

One embodiment is directed to a method for diagnosing a condition, syndrome or disease in a human associated with an influenza and/or RSV virus, comprising: (a) contacting a sample with at least one forward and reverse primer set selected from the group consisting of: Groups 1-38 of Table 3; (b) conducting an amplification reaction, thereby producing an amplicon; and (c) detecting the amplicon using one or more probes selected from the group consisting of: SEQ ID 2, 5 (influenza A); 10, 13, 16, 23, 25, 28, 30, 33, 39 and 42 (influenza B); and 45, 48, 51, 53, 72, 75, 78, 81, 84, 85, 86, 87 and 89 (RSV); wherein the generation of an amplicon is indicative of the presence of an influenza and/or RSV virus in the sample. In a particular embodiment, the sample is saliva, fluids collected from the ear, eye, mouth, and respiratory airways, sputum, tears, oropharyngeal swabs, nasopharyngeal swabs, nasal swabs, throat swabs, nasopharyngeal aspirates, bronchoalveolar lavage fluid, skin swabs, nasal aspirates, nasal wash, and fluids and cells obtained by the perfusion of tissues of both human and animal origin. In one embodiment, the sample is from a human, is non-human in origin, or is derived from an inanimate object or environmental surfaces. A sample may be collected from more than one collection site, e.g., oropharyngeal and nasopharyngeal swabs. In a particular embodiment, the complications, conditions, syndromes or diseases in humans associated with an influenza and/or RSV virus are selected from the group consisting of: asthma, middle ear infection, bronchiolitis, fever, chills, anorexia, headache, myalgia, weakness, sneezing, rhinitis, sore throat, a nonproductive cough, nausea, vomiting, pneumonia and death.

One embodiment is directed to a kit for amplifying and sequencing DNA of an influenza and/or RSV virus in a sample, comprising: (a) at least one forward primer or primer pair comprising the sequence selected from the group consisting of: SEQ ID NOS: 1, 4 and 7 (influenza A); 9, 12, 15, 19, 21, 22, 27, 32, 35, 38 and 41 (influenza B); and 44, 47, 50, 71, 74, 77, 80 and 88 (RSV)); and (b) at least one reverse primer or primer pair comprising the sequence selected from the group consisting of: SEQ ID NO: 3, 6 and 8 (influenza A); 11, 14, 17, 18, 20, 24, 26, 29, 31, 34, 36, 37, 40 and 43 (influenza B); and 46, 49, 52, 54, 55, 73, 76, 79, 82, 83 and 90 (RSV); and (c) reagents for the sequencing of amplified DNA fragments.

One embodiment is directed to a process control (MS2 bacteriophage, GI:15081). The MS2 bacteriophage is a well-characterized single-stranded RNA (ssRNA) virus of the Leviviridae family that is known to infect Enterobacteria, but does not occur naturally in clinical sample types for which this assay is intended. A target sequence in this process control is detected by a forward primer (SEQ ID NO: 56, 59, 62, 65 and 68), a reverse primer (SEQ ID NO: 58, 61, 64, 67 and 70) and a probe (SEQ ID NO: 57, 60, 63, 66 and 69). The process control is added directly to the clinical sample to monitor the integrity of both nucleic acid extraction/purification and PCR amplification steps.

Plasmids containing positive control sequences for one or more of the targets (i.e., Influenza A, Influenza B, RSV) are used for in vitro transcription of target ssRNA (IVT RNA) for the assays. IVT RNA comprising target RNA sequences serve as positive controls to confirm the assay is performing within specifications.

The oligonucleotides of the present invention and their resulting amplicons do not cross react and, thus, will work together without negatively impacting each other. The primers and probes to detect influenza A, influenza B and RSV do not cross react with each other. The primers and probes of the present invention do not cross react with other potentially contaminating species that would be present in a sample matrix.

### DETAILED DESCRIPTION

A diagnostic test or tests that can simultaneously detect and differentiate influenza A, influenza B and RSV is important, as respiratory infections are a primary health concern world-wide.

Described herein are optimized probes and primers that, alone or in various combinations, allow for the amplification, detection, differentiation, isolation, and sequencing of influenza and/or RSV viruses that can be found in clinical isolates. Specific probes and primers, i.e., probes and primers that can detect all known and characterized strains of influenza A, influenza B, and RSV, have been discovered and are described herein. Nucleic acid primers and probes for detecting specific influenza and/or RSV genetic material and methods for designing and optimizing the respective primer and probe sequences are described herein.

The primers and probes of the present invention can be used for the detection of influenza A, and/or influenza B and/or RSV, without loss of assay precision or sensitivity. The primers and probes described herein can be used, for example, to identify and/or confirm symptomatic patients for the presence of influenza and/or RSV viruses in a multiplex format.

### Influenza A and B

Influenza is a respiratory illness caused by influenza A or B viruses that occurs in outbreaks and epidemics worldwide. Influenza A viruses undergo periodic changes in the antigenic characteristics of their envelope glycoproteins, the hemagglutinin and the neuraminidase. Changes in these glycoproteins are referred to as antigenic shifts, which are associated with epidemics and pandemics of influenza A. There are three major subtypes of hemagglutinins (H1, H2, and H3) and two subtypes of neuraminidases (N1 and N2) among influenza A viruses that infect humans. There are two subtypes of influenza A, H1N1 or H3N2. Influenza B viruses are less likely to undergo antigenic changes. (Dolin, R. influenza In: Harrison's Principles of Process Medicine, 15th ed, Braunwald, E, Fauci, A S, Kasper, D L, et al. (Eds), McGraw Hill, New York, 2001, p. 1125). Influenza A outbreaks are usually seasonal and almost always occur during the winter months in the northern and southern hemispheres (which occur at different times of the year).

Symptoms of influenza include fever, headache, sore throat, myalgia, and weakness. Infection of influenza can be transmitted through sneezing and coughing via droplets and by contacting an animate or inanimate object that has flu virus on it. (Fiore A E; Shay D K; Broder K; Iskander J K; Uyeki T M; Mootrey G; Bresee J S; Cox N S, Prevention and Control of influenza: Recommendations of the Advisory Committee on Immunization Practices (ACIP), MMWR Recomm Rep. 2008 Aug. 8; 57(RR-7):1-60; Blachere F M; Lindsley W G; Pearce T A; Anderson S E; Fisher M; Khakoo R; Meade B J; Lander O; Davis S; Thewlis R E; Celik I; Chen B T; Beezhold D H, Measurement of Airborne influenza Virus in a Hospital Emergency Department, Clin Infect Dis. 2009 Jan. 9). Influenza virus shedding increases one-half to one day following exposure, peaking on the second day, then rapidly declines. The average duration of shedding is 4 to 5 days. Children, elderly adults, immunocompromised hosts and patients with chronic illnesses can shed the virus for longer periods of time. (Carrat F; Vergu E; Ferguson N M; Lemaitre M; Cauchemez S; Leach S; Valleron A J, Time lines of infection and disease in human influenza: a review of volunteer challenge studies, Am J Epidemiol. 2008 Apr. 1; 167(7):775-85. Epub 2008 Jan. 29; Leekha S; Zitterkopf N L; Espy M J; Smith T F; Thompson R L; Sampathkumar P, Duration of influenza A virus shedding in hospitalized patients and implications for infection control, Infect Control Hosp Epidemiol. 2007 Sep. 28; (9):1071-6).

Influenza infections may also have other presentations, such as afebrile respiratory illnesses. Complications of influenza include pneumonia, myositis and rhabdomyolysis, myalgias, central nervous system disease (CNS) including encephalitis, transverse myelitis, aseptic meningitis, and Guillain-Barré syndrome (GBS). (Bayer, W H. influenza B encephalitis. West J Med 1987; 147:466; Fujimoto S; Kobayashi M; Uemura O; Iwasa M; Ando T; Katoh T; Nakamura C; Maki N; Togari H; Wada Y, PCR on cerebrospinal fluid to show influenza-associated acute encephalopathy or encephalitis, Lancet 1998 Sep. 12; 352(9131):873-5).

### Respiratory Syncytial Virus

RSV causes acute lower respiratory infection among children and can also cause more serious diseases, including pneumonia. In 2005, an estimated 33.8 million new cases of acute lower respiratory infection associated with RSV occurred, mostly in developing countries. (Nair, H. et al. Global burden of acute lower respiratory infections due to respiratory syncytial virus in young children: a systematic review and meta analysis, Lancet, 2010, 375(9725): 1545-55). RSV infection occurs in almost all young children and studies indicate that RSV may persist in the respiratory tract, leading to reinfections. RSV is the most common cause of bronchiolitis among children less than 1 year old worldwide. RSV also can infect the elderly, transplant recipients and individuals afflicted with cystic fibrosis. (Eckardt-Michel, J; Lorek, M; Baxmann, D; Grunwald, T; Keil, G M; Zimmer, G, The fusion protein of respiratory syncytial virus triggers p53-dependent apoptosis, J. Virol. 2008 April: 82(7): 3236-3249).

Symptoms of RSV infection include a runny nose, decrease in appetite, wheezing, coughing, sneezing and fever. Droplets containing RSV emitted via sneezing or coughing in the air can spread the disease. The disease can be rapidly transmitted by direct and indirect contact with nasal or oral secretions. There is no known treatment for RSV, although the drug palivizumab has been shown to prevent severe RSV illness in some high-risk patients (www.cdc.gov/rsv, last accessed on Jul. 6, 2011).

### Assays

Table 1 demonstrates various possible diagnostic outcome scenarios using the probes and primers described herein in diagnostic methods.

TABLE 1

| Target | Expected Results | | | | | |
|---|---|---|---|---|---|---|
| Inf. A | + | + | + | − | + | − |
| Inf. B | + | + | − | − | − | + |
| RSV | + | − | − | − | + | − |
| MS2 | +/− | +/− | +/− | + | +/− | +/− |
| (PC) | | | | | | |
| Interpretation | Inf. A/ | Inf. A | Inf. | None | InfA/RSV | Inf. |
| | Inf. B/ | and | A | | | B/RSV |
| | RSV | Inf. B | | | | |

+, target detected;
−, target not detected;
Inf. A corresponding to the influenza A strain;
Inf. B corresponding to the influenza B strain;
RSV corresponding to Respiratory Syncytial Virus;
MS2 (PC) corresponding to the process control.

Detection of the process control (PC) indicates that the sample result is valid, where an absence of a signal corresponding to the PC indicates either an invalid result or that one or more of the specific targets is at a high starting concentration. A signal indicating a high starting concentration of specific target in the absence of a process control signal is considered to be a valid sample result.

The advantages of a multiplex format are: (1) simplified and improved testing and analysis; (2) increased efficiency and cost-effectiveness; (3) decreased turnaround time (increased speed of reporting results); (4) increased productivity (less equipment time needed); and (5) coordination/standardization of results for patients for multiple organisms (reduces error from inter-assay variation).

Detection of influenza and/or RSV can lead to earlier and more effective treatment of a subject. The methods for diagnosing and detecting influenza and/or RSV viruses described herein can be coupled with effective treatment therapies (e.g., antivirals). The treatments for such infections will depend upon the clinical disease state of the patient, as determinable by one of skill in the art.

The present invention therefore provides a method for specifically detecting in a sample the presence of two influenza types and respiratory syncytial virus using the primers and probes provided herein. Of particular interest in this regard is the ability of the disclosed primers and probes, as well as those that can be designed according to the disclosed methods, to specifically detect all or a majority of presently characterized strains of known, characterized influenza and RSV variants. The optimized primers and probes are useful, therefore, for identifying and diagnosing influenza and/or RSV infection, whereupon an appropriate treatment can then be administered to the individual to eradicate the virus(es).

The present invention provides one or more sets of primers that can anneal to all currently identified influenza A, influenza B and RSV strains and thereby amplify a target from a biological sample. The present invention provides, for example, at least a first primer and at least a second primer for influenza A, influenza B and RSV, each of which comprises a nucleotide sequence designed according to the inventive principles disclosed herein, which are used together to amplify DNA from influenza and/or RSV in a mixed-flora sample in a multiplex assay.

Also provided herein are probes that hybridize to the influenza and/or RSV sequences and/or amplified products derived from the influenza and/or RSV sequences. A probe can be labeled, for example, such that when it binds to an amplified or unamplified target sequence, or after it has been cleaved after binding, a fluorescent signal is emitted that is detectable under various spectroscopy and light measuring apparatuses. The use of a labeled probe, therefore, can enhance the sensitivity of detection of a target in an amplification reaction of DNA of influenza and/or RSV because it permits the detection of viral-derived DNA at low template concentrations that might not be conducive to visual detection as a gel-stained amplification product.

Primers and probes are sequences that anneal to a viral genomic or viral genomic derived sequence, e.g., the influenza strains (the “target” sequence). The target sequence can be, for example, an anti-viral resistance mutation or a viral genome. In one embodiment, the entire gene sequence can be “scanned” for optimized primers and probes useful for detecting the anti-viral resistance mutation or the viral genome. In other embodiments, particular regions of the genome can be scanned, e.g., regions that are documented in the literature as being useful for detecting multiple genes, regions that are conserved, or regions where sufficient information is available in, for example, a public database, with respect to the antibiotic resistance genes.

Sets or groups of primers and probes are generated based on the target to be detected. The set of all possible primers and probes can include, for example, sequences that include the variability at every site based on the known viral genome, or the primers and probes can be generated based on a consensus sequence of the target. The primers and probes are generated such that the primers and probes are able to anneal to a particular sequence under high stringency conditions. For example, one of skill in the art recognizes that for any particular sequence, it is possible to provide more than one oligonucleotide sequence that will anneal to the particular target sequence, even under high stringency conditions. The set of primers and probes to be sampled includes, for example, all such oligonucleotides for all known and characterized influenza viruses. Alternatively, the primers and probes include all such oligonucleotides for a given consensus sequence for a target.

Typically, stringent hybridization and washing conditions are used for nucleic acid molecules over about 500 bp. Stringent hybridization conditions include a solution comprising about 1 M Na⁺ at 25° C. to 30° C. below the Tm; e.g., 5×SSPE, 0.5% SDS, at 65° C.; see, Ausubel, et al., Current Protocols in Molecular Biology, Greene Publishing, 1995; Sambrook et al., Molecular Cloning: A Laboratory Manual, Cold Spring Harbor Press, 1989). Tm is dependent on both the G+C content and the concentration of salt ions, e.g., Na⁺ and K⁺. A formula to calculate the Tm of nucleic acid molecules greater than about 500 bp is Tm=81.5+0.41(% (G+C))−log₁₀[Na⁺]. Washing conditions are generally performed at least at equivalent stringency conditions as the hybridization. If the background levels are high, washing can be performed at higher stringency, such as around 15° C. below the Tm.

The set of primers and probes, once determined as described above, are optimized for hybridizing to a plurality of antibiotic resistance genes by employing scoring and/or ranking steps that provide a positive or negative preference or “weight” to certain nucleotides in a target nucleic acid strain sequence. If a consensus sequence is used to generate the full set of primers and probes, for example, then a particular primer sequence is scored for its ability to anneal to the corresponding sequence of every known native target sequence. Even if a probe were originally generated based on a consensus, the validation of the probe is in its ability to specifically anneal and detect every, or a large majority of, target sequences. The particular scoring or ranking steps performed depend upon the intended use for the primer and/or probe, the particular target nucleic acid sequence, and the number of resistance genes of that target nucleic acid sequence. The methods of the invention provide optimal primer and probe sequences because they hybridize to all or a subset of influenza and RSV viruses. Once optimized oligonucleotides are identified that can anneal to such genes, the sequences can then further be optimized for use, for example, in conjunction with another optimized sequence as a “primer set” or for use as a probe. A “primer set” is defined as at least one forward primer and one reverse primer.

Described herein are methods for using the primers and probes for producing a nucleic acid product, for example, comprising contacting one or more nucleic acid sequences of SEQ ID NOS: 1-55, 71-90 to a sample comprising the influenza and/or RSV strain under conditions suitable for nucleic acid polymerization. The primers and probes can additionally be used to sequence the DNA of the influenza type(s) and/or RSV, or used as diagnostics to, for example, detect the influenza type(s) and/or RSV in a clinical isolate sample, e.g., obtained from a subject, e.g., a mammalian subject. Particular combinations for amplifying DNA of influenza A, and/or influenza B, and/or RSV include, for example, using at least one forward primer selected from the group consisting of: SEQ ID NOS: 1, 4, 7, 9, 12, 15, 19, 21, 22, 27, 32, 35, 38, 41, 44, 47, 50, 56, 59, 62, 65, 68, 71, 74, 77, 80 and 88; and at least one reverse primer selected from the group consisting of SEQ ID NOS: 3, 6, 8, 11, 14, 17, 18, 20, 24, 26, 29, 31, 34, 36, 37, 40, 43, 46, 49, 52, 54, 55, 58, 61, 64, 67, 70, 73, 76, 79, 82, 83 and 90.

Methods are described for detecting influenza A, and/or influenza B, and/or RSV in a sample, for example, comprising (1) contacting at least one forward and reverse primer set, e.g., SEQ ID NOS: 1, 4, 7, 9, 12, 15, 19, 21, 22, 27, 32, 35, 38, 41, 44, 47, 50, 71, 74, 77, 80 and 88 (forward primers); and 3, 6, 8, 11, 14, 17, 18, 20, 24, 26, 29, 31, 34, 36, 37, 40, 43, 46, 49, 52, 54, 55, 73, 76, 79, 82, 83 and 90 (reverse primers) to a sample; (2) conducting an amplification; and (3) detecting the generation of an amplified product, wherein the generation of an amplified product indicates the presence of influenza A, and/or influenza B, and/or RSV pathogens in a clinical isolate sample.

The detection of amplicons using probes described herein can be performed, for example, using a labeled probe, e.g., the probe comprising a nucleotide sequence selected from the group consisting of: SEQ ID NOS: 2, 5, 10, 13, 16, 23, 25, 28, 30, 33, 39, 42, 45, 48, 51, 53, 72, 75, 78, 81, 84, 85, 86, 87 and 89 that hybridizes to one of the strands of the amplicon generated by at least one forward and reverse primer set. The probe(s) can be, for example, fluorescently labeled, thereby indicating that the detection of the probe involves measuring the fluorescence of the sample of the bound probe, e.g., after bound probes have been isolated. Probes can also be fluorescently labeled in such a way, for example, such that they only fluoresce upon hybridizing to their target, thereby eliminating the need to isolate hybridized probes. The probe can also comprise a fluorescent reporter moiety and a quencher of fluorescence moiety. Upon probe hybridization with the amplified product, the exonuclease activity of a DNA polymerase can be used to dissociate the probe's reporter and quencher, resulting in the unquenched emission of fluorescence, which is detected. An increase in the amplified product causes a proportional increase in fluorescence, due to cleavage of the probe and release of the reporter moiety of the probe. The amplified product is quantified in real time as it accumulates. For multiplex reactions involving more than one distinct probe, each of the probes can be labeled with a different distinguishable and detectable label.

The probes can be molecular beacons. Molecular beacons are single-stranded probes that form a stem-loop structure. A fluorophore can be, for example, covalently linked to one end of the stem and a quencher can be covalently linked to the other end of the stem forming a stem hybrid. When a molecular beacon hybridizes to a target nucleic acid sequence, the probe undergoes a conformational change that results in the dissociation of the stem hybrid and, thus the fluorophore and the quencher move away from each other, enabling the probe to fluoresce brightly. Molecular beacons can be labeled with differently colored fluorophores to detect different target sequences. Any of the probes described herein can be modified and utilized as molecular beacons.

The probes can be conjugated to a minor groove binder (MGB) group. This increases the stability of the probe template hybrid and reduces the tolerance for mismatches, which results in better discriminatory properties. With MGBs, the added functionality is due to a peptide moiety conjugated to the nucleic acid sequence that alters the binding properties of the probe.

The probes can alternatively be modified using locked nucleic acid (LNA) technology (see Kaur, H. et al., Biochemistry, 45:7347-55, 2006; and You, Y. et al., Nucl. Acids Res., 34:e60, 2006). LNA is a modified nucleic acid that is incorporated into the probe, replacing one or more of the nucleotides, thus altering the way that region of the probe binds to its complementary target sequence. A LNA, often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. The locked ribose conformation enhances base stacking and backbone pre-organization. This significantly increases the thermal stability (melting temperature) of oligonucleotides.

Primer or probe sequences can be ranked according to specific hybridization parameters or metrics that assign a score value indicating their ability to anneal to viral strains under highly stringent conditions. Where a primer set is being scored, a “first” or “forward” primer is scored and the “second” or “reverse”-oriented primer sequences can be optimized similarly but with potentially additional parameters, followed by an optional evaluation for primer dimers, for example, between the forward and reverse primers.

The scoring or ranking steps that are used in the methods of determining the primers and probes include, for example, the following parameters: a target sequence score for the target nucleic acid sequence(s), e.g., the PriMD® score; a mean conservation score for the target nucleic acid sequence(s); a mean coverage score for the target nucleic acid sequence(s); 100% conservation score of a portion (e.g., 5′ end, center, 3′ end) of the target nucleic acid sequence(s); a species score; a strain score; a subtype score; a serotype score; an associated disease score; a year score; a country of origin score; a duplicate score; a patent score; and a minimum qualifying score. Other parameters that are used include, for example, the number of mismatches, the number of critical mismatches (e.g., mismatches that result in the predicted failure of the sequence to anneal to a target sequence), the number of native strain sequences that contain critical mismatches, and predicted Tm values. The term “Tm” refers to the temperature at which a population of double-stranded nucleic acid molecules becomes half-dissociated into single strands. Methods for calculating the Tm of nucleic acids are known in the art (Berger and Kimmel (1987) Meth. Enzymol., Vol. 152: Guide To Molecular Cloning Techniques, San Diego: Academic Press, Inc. and Sambrook et al. (1989) Molecular Cloning: A Laboratory Manual, (2nd ed.) Vols. 1-3, Cold Spring Harbor Laboratory).

The resultant scores represent steps in determining nucleotide or whole target nucleic acid sequence preference, while tailoring the primer and/or probe sequences so that they hybridize to a plurality of target nucleic acid sequences. The methods of determining the primers and probes also can comprise the step of allowing for one or more nucleotide changes when determining identity between the candidate primer and probe sequences and the target nucleic acid sequences, or their complements.

In another embodiment, the methods of determining the primers and probes comprise the steps of comparing the candidate primer and probe nucleic acid sequences to “exclusion nucleic acid sequences” and then rejecting those candidate nucleic acid sequences that share identity with the exclusion nucleic acid sequences. In another embodiment, the methods comprise the steps of comparing the candidate primer and probe nucleic acid sequences to “inclusion nucleic acid sequences” and then rejecting those candidate nucleic acid sequences that do not share identity with the inclusion nucleic acid sequences.

In other embodiments of the methods of determining the primers and probes, optimizing primers and probes comprises using a polymerase chain reaction (PCR) penalty score formula comprising at least one of a weighted sum of: primer Tm—optimal Tm; difference between primer Tms; amplicon length—minimum amplicon length; and distance between the primer and a TagMan® probe. The optimizing step also can comprise determining the ability of the candidate sequence to hybridize with the most target nucleic acid strain sequences (e.g., the most target organisms or genes). In another embodiment, the selecting or optimizing step comprises determining which sequences have mean conservation scores closest to 1, wherein a standard of deviation on the mean conservation scores is also compared.

In other embodiments, the methods further comprise the step of evaluating which target nucleic acid sequences are hybridized by an optimal forward primer and an optimal reverse primer, for example, by determining the number of base pair differences between target nucleic acid sequences in a database. For example, the evaluating step can comprise performing an in silico polymerase chain reaction, involving (1) rejecting the forward primer and/or reverse primer if it does not meet inclusion or exclusion criteria; (2) rejecting the forward primer and/or reverse primer if it does not amplify a medically valuable nucleic acid; (3) conducting a BLAST analysis to identify forward primer sequences and/or reverse primer sequences that overlap with a published and/or patented sequence; and/or (4) determining the secondary structure of the forward primer, reverse primer, and/or target. In an embodiment, the evaluating step includes evaluating whether the forward primer sequence, reverse primer sequence, and/or probe sequence hybridizes to sequences in the database other than the nucleic acid sequences that are representative of the target strains.

The present invention provides oligonucleotides that have preferred primer and probe qualities. These qualities are specific to the sequences of the optimized probes, however, one of skill in the art would recognize that other molecules with similar sequences could also be used. The oligonucleotides provided herein comprise a sequence that shares at least about 60-70% identity with a sequence described in Table 3. In another embodiment, the invention provides a nucleic acid comprising a sequence that shares at least about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% identity with the sequences of Table 3 or complement thereof. The terms “homology” or “identity” or “similarity” refer to sequence relationships between two nucleic acid molecules and can be determined by comparing a nucleotide position in each sequence when aligned for purposes of comparison. The term “homology” refers to the relatedness of two nucleic acid or protein sequences. The term “identity” refers to the degree to which nucleic acids are the same between two sequences. The term “similarity” refers to the degree to which nucleic acids are the same, but includes neutral degenerate nucleotides that can be substituted within a codon without changing the amino acid identity of the codon, as is well known in the art.

In addition, the sequences, including those provided in Table 3 and sequences sharing certain sequence identities with those in Table 3, as described above, can be incorporated into longer sequences, provided they function to specifically anneal to and identify viral strains. In one aspect, the longer sequences have 1, 2, 3, 4, 5, 6, 7, 8, 9, or 10 additional bases at either or both ends of the original sequences. These longer sequences are also within the scope of the present disclosure.

The primer and/or probe nucleic acid sequences of the invention are complementary to the target nucleic acid sequence. The probe and/or primer nucleic acid sequences of the invention are optimal for identifying numerous strains of a target nucleic acid, e.g., influenza viruses and/or RSV. In an embodiment, the nucleic acids of the invention are primers for the synthesis (e.g., amplification) of target nucleic acid sequences and/or probes for identification, isolation, detection, or analysis of target nucleic acid sequences, e.g., an amplified target nucleic acid that is amplified using the primers of the invention.

The present oligonucleotides hybridize with more than one influenza type (as determined by differences in its sequence) and/or RSV. The probes and primers provided herein can, for example, allow for the detection of currently identified influenza types or a subset thereof as well as RSV variants. In addition, the primers and probes of the present invention, depending on the influenza sequence(s), can allow for the detection of previously unidentified influenza and RSV sequences. The methods of the invention provide for optimal primers and probes, and sets thereof, and combinations of sets thereof, which can hybridize with a larger number of targets than available primers and probes.

In other aspects, the invention also provides vectors (e.g., plasmid, phage, expression), cell lines (e.g., mammalian, insect, yeast, bacterial, viral), and kits comprising any of the sequences of the invention described herein. The invention further provides known or previously unknown target nucleic acid strain sequences that are identified, for example, using the methods of the invention. In an embodiment, the target nucleic acid sequence is an amplification product. In another embodiment, the target nucleic acid sequence is a native or synthetic nucleic acid. The primers, probes, and target nucleic acid sequences, vectors, cell lines, and kits can have any number of uses, such as diagnostic, investigative, confirmatory, monitoring, predictive or prognostic.

Diagnostic kits that comprise one or more of the oligonucleotides described herein, which are useful for screening for and/or detecting the presence of influenza and/or RSV in an individual and/or from a sample, are provided herein. An individual can be a human male, human female, human adult, human child, or human fetus. A sample includes any item, surface, material, clothing, or environment, in which it may be desirable to test for the presence of influenza virus(es) and/or RSV. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of influenza virus(es) and/or RSV.

A probe of the present invention can comprise a label such as, for example, a fluorescent label, a chemiluminescent label, a radioactive label, biotin, gold, dendrimers, aptamer, enzymes, proteins, quenchers and molecular motors. In an embodiment, the probe is a hydrolysis probe, such as, for example, a TagMan® probe. In other embodiments, the probes of the invention are molecular beacons, any fluorescent probes, probes modified with locked nucleic acids and probes that are replaced by any double stranded DNA binding dyes (e.g., SYBR® Green 1).

Oligonucleotides of the present invention do not only include primers that are useful for conducting the aforementioned amplification reactions, but also include oligonucleotides that are attached to a solid support, such as, for example, a microarray, multiwell plate, column, bead, glass slide, polymeric membrane, glass microfiber, plastic tubes, cellulose, and carbon nanostructures. Hence, detection of influenza viruses and/or RSV can be performed by exposing such an oligonucleotide-covered surface to a sample such that the binding of a complementary strain DNA sequence to a surface-attached oligonucleotide elicits a detectable signal or reaction.

Oligonucleotides of the present invention also include primers for isolating and sequencing nucleic acid sequences derived from any identified or yet to be isolated and identified influenza virus or RSV.

One embodiment of the invention uses solid support-based oligonucleotide hybridization methods to detect gene expression. Solid support-based methods suitable for practicing the present invention are widely known and are described (PCT application WO 95/11755; Huber et al., Anal. Biochem., 299:24, 2001; Meiyanto et al., Biotechniques, 31:406, 2001; Relogio et al., Nucleic Acids Res., 30:e51, 2002; the contents of which are incorporated herein by reference in their entirety). Any solid surface to which oligonucleotides can be bound, covalently or non-covalently, can be used. Such solid supports include, but are not limited to, filters, polyvinyl chloride dishes, silicon or glass based chips.

In certain embodiments, the nucleic acid molecule can be directly bound to the solid support or bound through a linker arm, which is typically positioned between the nucleic acid sequence and the solid support. A linker arm that increases the distance between the nucleic acid molecule and the substrate can increase hybridization efficiency. There are a number of ways to position a linker arm. In one common approach, the solid support is coated with a polymeric layer that provides linker arms with a plurality of reactive ends/sites. A common example of this type is glass slides coated with polylysine (U.S. Pat. No. 5,667,976, the contents of which are incorporated herein by reference in its entirety), which are commercially available. Alternatively, the linker arm can be synthesized as part of or conjugated to the nucleic acid molecule, and then this complex is bonded to the solid support. One approach, for example, takes advantage of the extremely high affinity biotin-streptavidin interaction. The streptavidin-biotinylated reaction is stable enough to withstand stringent washing conditions and is sufficiently stable that it is not cleaved by laser pulses used in some detection systems, such as matrix-assisted laser desorption/ionization time of flight (MALDI-TOF) mass spectrometry. Therefore, streptavidin can be covalently attached to a solid support, and a biotinylated nucleic acid molecule will bind to the streptavidin-coated surface. In one version of this method, an amino-coated silicon wafer is reacted with the n-hydroxysuccinimido-ester of biotin and complexed with streptavidin. Biotinylated oligonucleotides are bound to the surface at a concentration of about 20 fmol DNA per mm².

One can alternatively directly bind DNA to the support using carbodiimides, for example. In one such method, the support is coated with hydrazide groups, and then treated with carbodiimide. Carboxy-modified nucleic acid molecules are then coupled to the treated support. Epoxide-based chemistries are also being employed with amine modified oligonucleotides. Other chemistries for coupling nucleic acid molecules to solid substrates are known to those of skill in the art.

The nucleic acid molecules, e.g., the primers and probes of the present invention, must be delivered to the substrate material, which is suspected of containing or is being tested for the presence of influenza virus(es) and/or RSV. Because of the miniaturization of the arrays, delivery techniques must be capable of positioning very small amounts of liquids in very small regions, very close to one another and amenable to automation. Several techniques and devices are available to achieve such delivery. Among these are mechanical mechanisms (e.g., arrayers from GeneticMicroSystems, MA, USA) and ink-jet technology. Very fine pipets can also be used.

Other formats are also suitable within the context of this invention. For example, a 96-well format with fixation of the nucleic acids to a nitrocellulose or nylon membrane can also be employed.

After the nucleic acid molecules have been bound to the solid support, it is often useful to block reactive sites on the solid support that are not consumed in binding to the nucleic acid molecule. In the absence of the blocking step, excess primers and/or probes can, to some extent, bind directly to the solid support itself, giving rise to non-specific binding. Non-specific binding can sometimes hinder the ability to detect low levels of specific binding. A variety of effective blocking agents (e.g., milk powder, serum albumin or other proteins with free amine groups, polyvinylpyrrolidine) can be used and others are known to those skilled in the art (U.S. Pat. No. 5,994,065, the contents of which are incorporated herein by reference in their entirety). The choice depends at least in part upon the binding chemistry.

One embodiment uses oligonucleotide arrays, e.g., microarrays, that can be used to simultaneously observe the expression of a number of influenza viruses and/or RSV. Oligonucleotide arrays comprise two or more oligonucleotide probes provided on a solid support, wherein each probe occupies a unique location on the support. The location of each probe can be predetermined, such that detection of a detectable signal at a given location is indicative of hybridization to an oligonucleotide probe of a known identity. Each predetermined location can contain more than one molecule of a probe, but each molecule within the predetermined location has an identical sequence. Such predetermined locations are termed features. There can be, for example, from 2, 10, 100, 1,000, 2,000 or 5,000 or more of such features on a single solid support. In one embodiment, each oligonucleotide is located at a unique position on an array at least 2, at least 3, at least 4, at least 5, at least 6, or at least 10 times.

Oligonucleotide probe arrays for detecting gene expression can be made and used according to conventional techniques described (Lockhart et al., Nat. Biotech., 14:1675-1680, 1996; McGall et al., Proc. Natl. Acad. Sci. USA, 93:13555, 1996; Hughes et al., Nat. Biotechnol., 19:342, 2001). A variety of oligonucleotide array designs are suitable for the practice of this invention.

Generally, a detectable molecule, also referred to herein as a label, can be incorporated or added to an array's probe nucleic acid sequences. Many types of molecules can be used within the context of this invention. Such molecules include, but are not limited to, fluorochromes, chemiluminescent molecules, chromogenic molecules, radioactive molecules, mass spectrometry tags, proteins, and the like. Other labels will be readily apparent to one skilled in the art.

Oligonucleotide probes used in the methods of the present invention, including microarray techniques, can be generated using PCR. PCR primers used in generating the probes are chosen, for example, based on the sequences of Table 3. In one embodiment, oligonucleotide control probes also are used. Exemplary control probes can fall into at least one of three categories referred to herein as (1) normalization controls, (2) expression level controls and (3) negative controls. In microarray methods, one or more of these control probes can be provided on the array with the inventive viral oligonucleotides.

Normalization controls correct for dye biases, tissue biases, dust, slide irregularities, malformed slide spots, etc. Normalization controls are oligonucleotide or other nucleic acid probes that are complementary to labeled reference oligonucleotides or other nucleic acid sequences that are added to the nucleic acid sample to be screened. The signals obtained from the normalization controls, after hybridization, provide a control for variations in hybridization conditions, label intensity, reading efficiency and other factors that can cause the signal of a perfect hybridization to vary between arrays. The normalization controls also allow for the semi-quantification of the signals from other features on the microarray. In one embodiment, signals (e.g., fluorescence intensity or radioactivity) read from all other probes used in the method are divided by the signal from the control probes, thereby normalizing the measurements.

Virtually any probe can serve as a normalization control. Hybridization efficiency varies, however, with base composition and probe length. Preferred normalization probes are selected to reflect the average length of the other probes being used, but they also can be selected to cover a range of lengths. Further, the normalization control(s) can be selected to reflect the average base composition of the other probe(s) being used. In one embodiment, only one or a few normalization probes are used, and they are selected such that they hybridize well (i.e., without forming secondary structures) and do not match any test probes. In one embodiment, the normalization controls are mammalian genes.

“Negative control” probes are not complementary to any of the test oligonucleotides (i.e., the influenza and/or RSV oligonucleotides), normalization controls, or expression controls. In one embodiment, the negative control is a mammalian, viral or bacterial gene that is not complementary to any other sequence in the sample.

The terms “background” and “background signal intensity” refer to hybridization signals resulting from non-specific binding or other interactions between the labeled target nucleic acids (e.g., mRNA present in the biological sample) and components of the oligonucleotide array. Background signals also can be produced by intrinsic fluorescence of the array components themselves. A single background signal can be calculated for the entire array, or a different background signal can be calculated for each target nucleic acid. In one embodiment, background is calculated as the average hybridization signal intensity for the lowest 5 to 10 percent of the oligonucleotide probes being used, or, where a different background signal is calculated for each target gene, for the lowest 5 to 10 percent of the probes for each gene. Where the oligonucleotide probes corresponding to a particular target hybridize well and, hence, appear to bind specifically to a target sequence, they should not be used in a background signal calculation. Alternatively, background can be calculated as the average hybridization signal intensity produced by hybridization to probes that are not complementary to any sequence found in the sample (e.g., probes directed to nucleic acids of the opposite sense or to genes not found in the sample). In microarray methods, background can be calculated as the average signal intensity produced by regions of the array that lack any oligonucleotides probes at all.

In an alternative embodiment, the nucleic acid molecules are directly or indirectly coupled to an enzyme. Following hybridization, a chromogenic substrate is applied and the colored product is detected by a camera, such as a charge-coupled camera. Examples of such enzymes include alkaline phosphatase, horseradish peroxidase and the like. A probe can be labeled with an enzyme or, alternatively, the probe is labeled with a moiety that is capable of binding to another moiety that is linked to the enzyme. For example, in the biotin-streptavidin interaction, the streptavidin is conjugated to an enzyme such as horseradish peroxidase (HRP). A chromogenic substrate is added to the reaction and is processed/cleaved by the enzyme. The product of the cleavage forms a color, either in the UV or visible spectrum. In another embodiment, streptavidin alkaline phosphatase can be used in a labeled streptavidin-biotin immunoenzymatic antigen detection system.

The invention also provides methods of labeling nucleic acid molecules with cleavable mass spectrometry tags (CMST; U.S. Patent Application No. 60/279,890). After an assay is complete, and the uniquely CMST-labeled probes are distributed across the array, a laser beam is sequentially directed to each member of the array. The light from the laser beam both cleaves the unique tag from the tag-nucleic acid molecule conjugate and volatilizes it. The volatilized tag is directed into a mass spectrometer. Based on the mass spectrum of the tag and knowledge of how the tagged nucleotides were prepared, one can unambiguously identify the nucleic acid molecules to which the tag was attached (WO 9905319).

The nucleic acids, primers and probes of the present invention can be labeled readily by any of a variety of techniques. When the diversity panel is generated by amplification, the nucleic acids can be labeled during the reaction by incorporation of a labeled dNTP or use of labeled amplification primer. If the amplification primers include a promoter for an RNA polymerase, a post-reaction labeling can be achieved by synthesizing RNA in the presence of labeled NTPs. Amplified fragments that were unlabeled during amplification or unamplified nucleic acid molecules can be labeled by one of a number of end labeling techniques or by a transcription method, such as nick-translation, random-primed DNA synthesis. Details of these methods are known to one of skill in the art and are set out in methodology books. Other types of labeling reactions are performed by denaturation of the nucleic acid molecules in the presence of a DNA-binding molecule, such as RecA, and subsequent hybridization under conditions that favor the formation of a stable RecA-incorporated DNA complex.

In another embodiment, PCR-based methods are used to detect gene expression. These methods include reverse-transcriptase-mediated polymerase chain reaction (RT-PCR) including real-time and endpoint quantitative reverse-transcriptase-mediated polymerase chain reaction (Q-RTPCR). These methods are well known in the art. For example, methods of quantitative PCR can be carried out using kits and methods that are commercially available from, for example, Applied BioSystems and Stratagene®. See also Kochanowski, Quantitative PCR Protocols (Humana Press, 1999); Innis et al., supra.; Vandesompele et al., Genome Biol., 3: RESEARCH 0034, 2002; Stein, Cell Mol. Life Sci. 59:1235, 2002.

The forward and reverse amplification primers and internal hybridization probe is designed to hybridize specifically and uniquely with one nucleotide sequence derived from the transcript of a target gene. In one embodiment, the selection criteria for primer and probe sequences incorporates constraints regarding nucleotide content and size to accommodate TaqMan® requirements. SYBR® Green can be used as a probe-less Q-RTPCR alternative to the TaqMan®-type assay, discussed above (ABI Prism® 7900 Sequence Detection System User Guide Applied Biosystems, chap. 1-8, App. A-F. (2002)). A device measures changes in fluorescence emission intensity during PCR amplification. The measurement is done in “real time,” that is, as the amplification product accumulates in the reaction. Other methods can be used to measure changes in fluorescence resulting from probe digestion. For example, fluorescence polarization can distinguish between large and small molecules based on molecular tumbling (U.S. Pat. No. 5,593,867).

The primers and probes of the present invention may anneal to or hybridize to various influenza and/or RSV genetic material or genetic material derived therefrom, or other genetic material derived therefrom, such as RNA, DNA, cDNA, or a PCR product.

A “sample” that is tested for the presence of influenza virus(es) and/or RSV includes, but is not limited to a tissue sample, such as, for example, saliva, fluids collected from the ear, eye, mouth, and respiratory airways, sputum, skin, tears, oropharyngeal swabs, nasopharyngeal swabs, nasal swabs, throat swabs, skin swabs, nasal aspirates, and nasal wash. The tissue sample may be fresh, fixed, preserved, or frozen. A sample also includes any item, surface, material, or clothing, or environment, in which it may be desirable to test for the presence of influenza virus(es) and/or RSV. Thus, for instance, the present invention includes testing door handles, faucets, table surfaces, elevator buttons, chairs, toilet seats, sinks, kitchen surfaces, children's cribs, bed linen, pillows, keyboards, and so on, for the presence of influenza virus(es) and/or RSV.

The target nucleic acid strain that is amplified may be RNA or DNA or a modification thereof. Thus, the amplifying step can comprise isothermal or non-isothermal reactions, such as polymerase chain reaction, Scorpion® primers, molecular beacons, SimpleProbes®, HyBeacons®, cycling probe technology, Invader Assay, self-sustained sequence replication, nucleic acid sequence-based amplification, ramification amplifying method, hybridization signal amplification method, rolling circle amplification, multiple displacement amplification, thermophilic strand displacement amplification, transcription-mediated amplification, ligase chain reaction, signal mediated amplification of RNA, split promoter amplification, Q-Beta replicase, isothermal chain reaction, one cut event amplification, loop-mediated isothermal amplification, molecular inversion probes, ampliprobe, headloop DNA amplification, and ligation activated transcription. The amplifying step can be conducted on a solid support, such as a multiwell plate, array, column, bead, glass slide, polymeric membrane, glass microfiber, plastic tubes, cellulose, and carbon nanostructures. The amplifying step also comprises in situ hybridization. The detecting step can comprise gel electrophoresis, fluorescence resonant energy transfer, or hybridization to a labeled probe, such as a probe labeled with biotin, at least one fluorescent moiety, an antigen, a molecular weight tag, and a modifier of probe Tm. The detection step can also comprise the incorporation of a label (e.g., fluorescent or radioactive) during an extension reaction. The detecting step comprises measuring fluorescence, mass, charge, and/or chemiluminescence.

The target nucleic acid strain may not need amplification and may be RNA or DNA or a modification thereof. If amplification is not necessary, the target nucleic acid strain can be denatured to enable hybridization of a probe to the target nucleic acid sequence.

Hybridization may be detected in a variety of ways and with a variety of equipment. In general, the methods can be categorized as those that rely upon detectable molecules incorporated into the diversity panels and those that rely upon measurable properties of double-stranded nucleic acids (e.g., hybridized nucleic acids) that distinguish them from single-stranded nucleic acids (e.g., unhybridized nucleic acids). The latter category of methods includes intercalation of dyes, such as, for example, ethidium bromide, into double-stranded nucleic acids, differential absorbance properties of double and single stranded nucleic acids, binding of proteins that preferentially bind double-stranded nucleic acids, and the like.

### EXEMPLIFICATION

### Example 1

### Scoring a Set of Predicted Annealing Oligonucleotides

Each of the sets of primers and probes selected is ranked by a combination of methods as individual primers and probes and as a primer/probe set. This involves one or more methods of ranking (e.g., joint ranking, hierarchical ranking, and serial ranking) where sets of primers and probes are eliminated or included based on any combination of the following criteria, and a weighted ranking again based on any combination of the following criteria, for example: (A) Percentage Identity to Target Strains; (B) Conservation Score; (C) Coverage Score; (D) Strain/Subtype/Serotype Score; (E) Associated Disease Score; (F) Duplicates Sequences Score; (G) Year and Country of Origin Score; (H) Patent Score, and (I) Epidemiology Score.

### (A) Percentage Identity

A percentage identity score is based upon the number of target nucleic acid strain (e.g., native) sequences that can hybridize with perfect conservation (the sequences are perfectly complimentary) to each primer or probe of a primer set and probe set. If the score is less than 100%, the program ranks additional primer set and probe sets that are not perfectly conserved. This is a hierarchical scale for percent identity starting with perfect complimentarity, then one base degeneracy through to the number of degenerate bases that would provide the score closest to 100%. The position of these degenerate bases would then be ranked. The methods for calculating the conservation is described under section B.

(i) Individual Base Conservation Score

A set of conservation scores is generated for each nucleotide base in the consensus sequence and these scores represent how many of the target nucleic acid strains sequences have a particular base at this position. For example, a score of 0.95 for a nucleotide with an adenosine, and 0.05 for a nucleotide with a cytidine means that 95% of the native sequences have an A at that position and 5% have a C at that position. A perfectly conserved base position is one where all the target nucleic acid strain sequences have the same base (either an A, C, G, or T/U) at that position. If there is an equal number of bases (e.g., 50% A & 50% T) at a position, it is identified with an N.

(ii) Candidate Primer/Probe Sequence Conservation

An overall conservation score is generated for each candidate primer or probe sequence that represents how many of the target nucleic acid strain sequences will hybridize to the primers or probes. A candidate sequence that is perfectly complimentary to all the target nucleic acid strain sequences will have a score of 1.0 and rank the highest. For example, illustrated below in Table 2 are three different 10-base candidate probe sequences that are targeted to different regions of a consensus target nucleic acid strain sequence. Each candidate probe sequence is compared to a total of 10 native sequences.

TABLE 2

| | | | | | | | | | | |
|---|---|---|---|---|---|---|---|---|---|---|
| #1. | A | A | A | C | A | C | G | T | G | C |
| | 0.7 | 1.0 | 1.0 | 1.0 | 1.0 | 1.0 | 1.0 | 1.0 | 1.0 | 1.0 |

(SEQ ID NO: 91)
→Number of target nucleic acid strain sequences that are perfectly
complimentary - 7. Three out of the ten sequences do not have an A at
position 1.

| | | | | | | | | | | |
|---|---|---|---|---|---|---|---|---|---|---|
| #2. | C | C | T | T | G | T | T | C | C | A |
| | 1.0 | 0.9 | 1.0 | 0.9 | 0.9 | 1.0 | 1.0 | 1.0 | 1.0 | 1.0 |

(SEQ ID NO: 92)
→Number of target nucleic acid strain sequences that are perfectly
complimentary - 7, 8, or 9. At least one target nucleic acid strain does
not have a C at position 2, T at position 4, or G at position 5. These
differences may all be on one target nucleic acid strain molecule or may
be on two or three separate molecules.

| | | | | | | | | | | |
|---|---|---|---|---|---|---|---|---|---|---|
| #3. | C | A | G | G | G | A | C | G | A | T |
| | 1.0 | 1.0 | 1.0 | 1.0 | 1.0 | 0.9 | 0.8 | 1.0 | 1.0 | 1.0 |

(SEQ ID NO: 93)
→Number of target nucleic acid strain sequences that are perfectly
complimentary - 7 or 8. At least one target nucleic acid strain does not
have an A at position 6 and at least two target nucleic acid strain do not
have a C at position 7. These differences may all be on one target
nucleic acid strain molecule or may be on two separate molecules.

A simple arithmetic mean for each candidate sequence would generate the same value of 0.97. The number of target nucleic acid strain sequences identified by each candidate probe sequence, however, can be very different. Sequence #1 can only identify 7 native sequences because of the 0.7 (out of 1.0) score by the first base—A. Sequence #2 has three bases each with a score of 0.9; each of these could represent a different or shared target nucleic acid strain sequence. Consequently, Sequence #2 can identify 7, 8 or 9 target nucleic acid strain sequences. Similarly, Sequence #3 can identify 7 or 8 of the target nucleic acid strain sequences. Sequence #2 would, therefore, be the best choice if all the three bases with a score of 0.9 represented the same 9 target nucleic acid strain sequences.

(iii) Overall Conservation Score of the Primer and Probe Set—Percent Identity

The same method described in (ii) when applied to the complete primer set and probe set will generate the percent identity for the set (see A above). For example, using the same sequences illustrated above, if Sequences #1 and #2 are primers and Sequence #3 is a probe, then the percent identity for the target can be calculated from how many of the target nucleic acid sequences are identified with perfect complementarity to all three primer/probe sequences. The percent identity could be no better than 0.7 (7 out of 10 target nucleic acid strain sequences) but as little as 0.1 if each of the degenerate bases reflects a different target nucleic acid strain sequence. Again, an arithmetic mean of these three sequences would be 0.97. As none of the above examples were able to capture all the target nucleic acid strain sequences because of the degeneracy (scores of less than 1.0), the ranking system takes into account that a certain amount of degeneracy can be tolerated under normal hybridization conditions, for example, during a polymerase chain reaction. The ranking of these degeneracies is described in (iv) below.

An in silico evaluation determines how many native sequences (e.g., original sequences submitted to public databases) are identified by a given candidate primer/probe set. The ideal candidate primer/probe set is one that can perform PCR and the sequences are perfectly complementary to all the known native sequences that were used to generate the consensus sequence. If there is no such candidate, then the sets are ranked according to how many degenerate bases can be accepted and still hybridize to just the target sequence during the PCR and yet identify all the native sequences.

The hybridization conditions, for TagMan® as an example, are: 10-50 mM Tris-HCl pH 8.3, 50 mM KCl, 0.1-0.2% Triton® X-100 or 0.1% Tween®, 1-5 mM MgCl₂. The hybridization is performed at 58-60° C. for the primers and 68-70° C. for the probe. The in silico PCR identifies native sequences that are not amplifiable using the candidate primers and probe set. The rules can be as simple as counting the number of degenerate bases to more sophisticated approaches based on exploiting the PCR criteria used by the PriMD® software. Each target nucleic acid strain sequence has a value or weight (see Score assignment above). If the failed target nucleic acid strain sequence is medically valuable, the primer/probe set is rejected. This in silico analysis provides a degree of confidence for a given genotype and is important when new sequences are added to the databases. New target nucleic acid strain sequences are automatically entered into both the “include” and “exclude” categories. Published primer and probes will also be ranked by the PriMD software.

(iv) Position (5′ to 3′) of the Base Conservation Score

In an embodiment, primers do not have bases in the terminal five positions at the 3′ end with a score less than 1. This is one of the last parameters to be relaxed if the method fails to select any candidate sequences. The next best candidate having a perfectly conserved primer would be one where the poorer conserved positions are limited to the terminal bases at the 5′ end. The closer the poorer conserved position is to the 5′ end, the better the score. For probes, the position criteria are different. For example, with a TagMan® probe, the most destabilizing effect occurs in the center of the probe. The 5′ end of the probe is also important as this contains the reporter molecule that must be cleaved, following hybridization to the target, by the polymerase to generate a sequence-specific signal. The 3′ end is less critical. Therefore, a sequence with a perfectly conserved middle region will have the higher score. The remaining ends of the probe are ranked in a similar fashion to the 5′ end of the primer. Thus, the next best candidate to a perfectly conserved TagMan® probe would be one where the poorer conserved positions are limited to the terminal bases at either the 5′ or 3′ ends. The hierarchical scoring will select primers with only one degeneracy first, then primers with two degeneracies next and so on. The relative position of each degeneracy will then be ranked favoring those that are closest to the 5′ end of the primers and those closest to the 3′ end of the TagMan® probe. If there are two or more degenerate bases in a primer and probe set the ranking will initially select the sets where the degeneracies occur on different sequences.

### B. Coverage Score

The total number of aligned sequences is considered under a coverage score. A value is assigned to each position based on how many times that position has been reported or sequenced. Alternatively, coverage can be defined as how representative the sequences are of the known strains, subtypes etc., or their relevance to a certain diseases. For example, the target nucleic acid strain sequences for a particular gene may be very well conserved and show complete coverage but certain strains are not represented in those sequences.

A sequence is included if it aligns with any part of the consensus sequence, which is usually a whole gene or a functional unit, or has been described as being a representative of this gene. Even though a base position is perfectly conserved it may only represent a fraction of the total number of sequences (for example, if there are very few sequences). For example, region A of a gene shows a 100% conservation from 20 sequence entries while region B in the same gene shows a 98% conservation but from 200 sequence entries. There is a relationship between conservation and coverage if the sequence shows some persistent variability. As more sequences are aligned, the conservation score falls, but this effect is lessened as the number of sequences gets larger. Unless the number of sequences is very small (e.g., under 10) the value of the coverage score is small compared to that of the conservation score. To obtain the best consensus sequence, artificial spaces are allowed to be introduced. Such spaces are not considered in the coverage score.

### C. Strain/Subtype/Serotype Score

A value is assigned to each strain or subtype or serotype based upon its relevance to a disease. For example, viral strains and/or species that are linked to high frequencies of infection will have a higher score than strains that are generally regarded as benign. The score is based upon sufficient evidence to automatically associate a particular strain with a disease. For example, certain strains of adenovirus are not associated with diseases of the upper respiratory system. Accordingly, there will be sequences included in the consensus sequence that are not associated with diseases of the upper respiratory system.

### D. Associated Disease Score

The associated disease score pertains to strains that are not known to be associated with a particular disease (to differentiate from D above). Here, a value is assigned only if the submitted sequence is directly linked to the disease and that disease is pertinent to the assay.

### E. Duplicate Sequences Score

If a particular sequence has been sequenced more than once it will have an effect on representation, for example, a strain that is represented by 12 entries in GenBank of which six are identical and the other six are unique. Unless the identical sequences can be assigned to different strains/subtypes (usually by sequencing other gene or by immunology methods) they will be excluded from the scoring.

### F. Year and Country of Origin Score

The year and country of origin scores are important in terms of the age of the human population and the need to provide a product for a global market. For example, strains identified or collected many years ago may not be relevant today. Furthermore, it is probably difficult to obtain samples that contain these older strains. Certain divergent strains from more obscure countries or sources may also be less relevant to the locations that will likely perform clinical tests, or may be more important for certain countries (e.g., North America, Europe, or Asia).

### G. Patent Score

Candidate target strain sequences published in patents are searched electronically and annotated such that patented regions are excluded. Alternatively, candidate sequences are checked against a patented sequence database.

### H. Minimum Qualifying Score

The minimum qualifying score is determined by expanding the number of allowed mismatches in each set of candidate primers and probes until all possible native sequences are represented (e.g., has a qualifying hit).

### I. Other

A score is given based on other parameters, such as relevance to certain patients (e.g., pediatrics, immunocompromised) or certain therapies (e.g., target those strains that respond to treatment) or epidemiology. The prevalence of an organism/strain and the number of times it has been tested for in the community can add value to the selection of the candidate sequences. If a particular strain is more commonly tested then selection of it would be more likely. Strain identification can be used to select better vaccines.

### Example 2

### Primer/Probe Evaluation

Once the candidate primers and probes have received their scores and have been ranked, they are evaluated using any of a number of methods of the invention, such as BLAST analysis and secondary structure analysis.

### A. BLAST Analysis

The candidate primer/probe sets are submitted to BLAST analysis to check for possible overlap with any published sequences that might be missed by the Include/Exclude function. It also provides a useful summary.

### B. Secondary Structure

The methods of the present invention include analysis of nucleic acid secondary structure. This includes the structures of the primers and/or probes, as well as their intended target strain sequences. The methods and software of the invention predict the optimal temperatures for annealing, but assumes that the target (e.g., RNA or DNA) does not have any significant secondary structure. For example, if the starting material is RNA, the first stage is the creation of a complimentary strand of DNA (cDNA) using a specific primer. This is usually performed at temperatures where the RNA template can have significant secondary structure thereby preventing the annealing of the primer. Similarly, after denaturation of a double stranded DNA target (for example, an amplicon after PCR), the binding of the probe is dependent on there being no major secondary structure in the amplicon.

The methods of the invention can either use this information as a criteria for selecting primers and probes or evaluate any secondary structure of a selected sequence, for example, by cutting and pasting candidate primer or probe sequences into a commercial internet link that uses software dedicated to analyzing secondary structure, such as, for example, MFOLD (Zuker et al. (1999) Algorithms and Thermodynamics for RNA Secondary Structure Prediction: A Practical Guide in RNA Biochemistry and Biotechnology, J. Barciszewski and B. F. C. Clark, eds., NATO ASI Series, Kluwer Academic Publishers).

### C. Evaluating the Primer and Probe Sequences

The methods and software of the invention may also analyze any nucleic acid sequence to determine its suitability in a nucleic acid amplification-based assay. For example, it can accept a competitor's primer set and determine the following information: (1) How it compares to the primers of the invention (e.g., overall rank, PCR and conservation ranking, etc.); (2) How it aligns to the exclude libraries (e.g., assessing cross-hybridization)—also used to compare primer and probe sets to newly published sequences; and (3) If the sequence has been previously published. This step requires keeping a database of sequences published in scientific journals, posters, and other presentations.

### Example 3

### Multiplexing

The Exclude/Include capability is ideally suited for designing multiplex reactions. The parameters for designing multiple primer and probe sets adhere to a more stringent set of parameters than those used for the initial Exclude/Include function. Each set of primers and probe, together with the resulting amplicon, is screened against the other sets that constitute the multiplex reaction. As new targets are accepted, their sequences are automatically added to the Exclude category.

The database is designed to interrogate the online databases to determine and acquire, if necessary, any new sequences relevant to the targets. These sequences are evaluated against the optimal primer/probe set. If they represent a new genotype or strain, then a multiple sequence alignment may be required.

### Example 4

### Sequences Identified for Detecting Influenza A and/or Influenza B and/or RSV

The set of primers and probes were then scored according to the methods described herein to identify the optimized primers and probes of Table 3. It should be noted that the primers, as they are sequences that anneal to a plurality of identified or unidentified influenza A, influenza B and RSV, can also be used as probes either in the presence or absence of amplification of a sample.

TABLE 3
Optimized Primers and Probes for the Detection of Influenza A, Influenza B, RSV,
and Process Control.

| | | | |
|---|---|---|---|
| Group | | | |
| No. | Forward Primer | Probe | Reverse Primer |

Influenza A

| | | | |
|---|---|---|---|
| 1 | GCTCTCATGGAATGGCTAAAGAC | TCACCGTGCCCAGTGAGCGAG | GCATTTTGGACAAAGCGTCTACG |
| | SEQ ID NO: 1 | SEQ ID NO: 2 | SEQ ID NO: 3 |
| 2 | GGGATTTTGGGATTTGTGTTCACGCTCAC | TACGCTGCAGTCCTCGCTCAGTGGGC | TTCCCATTAAGGGCATTTTGGACAAA |
| | | ACG | GCG |
| | SEQ ID NO: 4 | SEQ ID NO: 5 | SEQ ID NO: 6 |
| | TAAAGACAAGACCAATCTTGTCACCTC | | TTACCATTGAGGGCATTTTGGACAAA |
| | TGACTAAGGG | | GCG |
| | SEQ ID NO: 7 | | SEQ ID NO: 8 |

Influenza B

| | | | |
|---|---|---|---|
| 3 | TTACAGTGGAGGATGAAGAAGATG | CATTAAGACGCTCGAAGAGTGAATTGA | CTCGAATTGGCTTTGAATGT |
| | SEQ ID NO: 9 | GGA | SEQ ID NO: 11 |
| | | SEQ ID NO: 10 | |
| 4 | TGGATACAAGTCCTTATCAACTCTG | TCGAAGAGTGAGTTGAGGATCCG | TGCTCTTGACCAAATTGGGAT |
| | SEQ ID NO: 12 | SEQ ID NO: 13 | SEQ ID NO: 14 |
| | GTTGCTAAACTTGTTGCTACTGA | TTGAGGATCCGATGGCCATCTT | GCTGCTCGAATTGGCTTT |
| | SEQ ID NO: 15 | SEQ ID NO: 16 | SEQ ID NO: 17 |
| 5 | TGGATACAAGTCCTTATCAACTCTG | TCGAAGAGTGAGTTGAGGATCCGG | TGGTGATAATCGGTGCTCTTG |
| | SEQ ID NO: 12 | SEQ ID NO: 13 | SEQ ID NO: 18 |
| | GCTAAACTTGTTGCTACTGATGA | TTGAGGATCCGATGGCCATCTT | GCTGCTCGAATTGGCTTT |
| | SEQ ID NO: 19 | SEQ ID NO: 16 | SEQ ID NO: 17 |
| 6 | TGGATACAAGTCCTTATCAACTCTG | TCGAAGAGTGAGTTGAGGATCCG | TGCTCTTGACCAAATTGGGAT |
| | SEQ ID NO: 12 | SEQ ID NO: 13 | SEQ ID NO: 14 |
| | GCTAAACTTGTTGCTACTGATGA | TTGAGGATCCGATGGCCATCTT | GCTGCTCGAATTGGCTTT |
| | SEQ ID NO: 19 | SEQ ID NO: 16 | SEQ ID NO: 17 |
| 7 | TGGATACAAGTCCTTATCAACTCTG | TCGAAGAGTGAGTTGAGGATCCG | TGGTGATAATCGGTGCTCTTG |
| | SEQ ID NO: 12 | SEQ ID NO: 13 | SEQ ID NO: 18 |
| | GTTGCTAAACTTGTTGCTACTGA | TTGAGGATCCGATGGCCATCTT | GCTGCTCGAATTGGCTTT |
| | SEQ ID NO: 15 | SEQ ID NO: 16 | SEQ ID NO: 17 |
| 8 | TGGATACAAGTCCTTATCAACTCTG | TCGAAGAGTGAGTTGAGGATCCG | TCGGTGCTCTTGACCAAATT |
| | SEQ ID NO: 12 | SEQ ID NO: 13 | SEQ ID NO: 20 |
| | GTTGCTAAACTTGTTGCTACTGA | TTGAGGATCCGATGGCCATCTT | GCTGCTCGAATTGGCTTT |
| | SEQ ID NO: 15 | SEQ ID NO: 16 | SEQ ID NO: 17 |
| 9 | TACAAGTCCTTATCAACTCTGCAT | TCGAAGAGTGAGTTGAGGATCCG | TGGTATAATCGGTGCTCTTG |
| | SEQ ID NO: 21 | SEQ ID NO: 13 | SEQ ID NO: 18 |
| | GTTGCTAAACTTGTTGCTACTGA | TTGAGGATCCGATGGCCATCTT | GCTGCTCGAATTGGCTTT |
| | SEQ ID NO: 15 | SEQ ID NO: 16 | SEQ ID NO: 17 |
| 10 | CTTGTTGCTAAACTTGTTGC | TCGGATCCTCAACTCACTCTTCG | TCAGCTGCTCGAATTG |
| | SEQ ID NO: 22 | SEQ ID NO: 23 | SEQ ID NO: 24 |
| | | TCGGATCCTCAATTCACTCTTCG | TTTCAGCTGCTCGAATTG |
| | | SEQ ID NO: 25 | SEQ ID NO: 26 |
| 11 | TGGATACAAGTCCTTATCAACTCTG | TTGAGGATCCGATGGCCATCTT | GCTGCTCGAATTGGCTTT |
| | SEQ ID NO: 12 | SEQ ID NO: 16 | SEQ ID NO: 17 |
| | GTTGCTAAACTTGTTGCTACTGA | | |
| | SEQ ID NO: 15 | | |
| 12 | CATCGGATCCTCAATTCACTCTTCG | AATGAAGGACATTCAAAGCCAATTCGA | CTTGACCAAATTGGGATAAGACTCC |
| | SEQ ID NO: 27 | GCAGCTGA | GCAGCTGASEQ ID NO: 29 |
| | | SEQ ID NO: 28 | |
| 13 | CATCGGATCCTCAATTCACTCTTCG | CAAAGCCAATTCGAGCAGCTGAAACTG | CTTGACCAAATTGGGATAAGACTCC |
| | SEQ ID NO: 27 | CG | SEQ ID NO: 29 |
| | | SEQ ID NO: 30 | |
| 14 | CATCGGATCCTCAATTCACTCTTCG | CAAAGCCAATTCGAGCAGCTGAAACTG | GTGATAATCGGTGCTCTTGACCAAA |
| | SEQ ID NO: 27 | CG | SEQ ID NO: 31 |
| | | SEQ ID NO: 30 | |
| 15 | CATCGGATCCTCAATTCACTCTTCG | AATGAAGGACATTCAAAGCCAATTCGA | GTGATAATCGGTGCTCTTGACCAAA |
| | SEQ ID NO: 27 | GCAGCTGA | SEQ ID NO: 31 |
| | | SEQ ID NO: 28 | |
| 16 | AACATGACCACAACACAAATTGAGG | TCCTGCTTCAAAGTTTATAGTGGCATTG | GTAATCAAGGGCTCTTTGCCATGAA |
| | SEQ ID NO: 32 | GTTGCTC | SEQ ID NO: 34 |
| | | SEQ ID NO: 33 | |
| | TCACAACACAAATTGAGGTGGGT | | TTGGCCAGGGTAGTCAAGGG |
| | SEQ ID NO: 35 | | SEQ ID NO: 36 |
| 17 | AACATGACCACAACACAAATTGAGG | TCCTGCTTCAAAGTTTATAGTGGCATTG | CTGTTTAGGCGGTTTTGACAG |
| | SEQ ID NO: 32 | GTTGCTC | SEQ ID NO: 37 |
| | | SEQ ID NO: 33 | |
| | TCACAACACAAATTGAGGTGGGT | | GTAATCAAGGGCTCTTTGCCATGAA |
| | SEQ ID NO: 35 | | SEQ ID NO: 34 |
| 18 | GTTGCTAAACTTGTTGCTACTGATGATC | AGAGCGCTCGAAGAGTGAGTTGAGGAT | GCTGCTCGAATTGGTTTTGAATGTCC |
| | TTACAGTGGAG | CCGATGGCC | TTCAT |
| | SEQ ID NO: 38 | SEQ ID NO: 39 | SEQ ID NO: 40 |
| 19 | GTTGCTAAACTTGTTGCTACTGATGATC | AGACGCTCGAAGAGTGAGTTGAGGAT | GCTGCTCGAATTGGTTTTGAATGTCC |
| | TTACAGTGGAG | CCGATGGC | TTCAT |
| | SEQ ID NO: 41 | SEQ ID NO: 42 | SEQ ID NO: 43 |

Respiratory Syncytial Virus (RSV)

| | | | |
|---|---|---|---|
| 20 | CCACCAACATCAAAGAAGGATCAAA | TGATCCTGCATTGTCACAGTACCATC | CTTTACAAGTGTCAGCCTGTGG |
| | SEQ ID NO: 44 | T | SEQ ID NO: 46 |
| | | SEQ ID NO: 45 | |
| 21 | TCCCCTCTATGTACAACCAACACAA | TGATCCTGCATTATCACAATACCATCCT | CTTTACATGTTCAGCTTGTGGGA |
| | SEQ ID NO: 47 | SEQ ID NO: 48 | SEQ ID NO: 49 |
| 22 | CCTCTTGTCACAATAATATGTACATATA | TGACACCACCCTTCGATACCACCCATG | GTGATATAGCTTCTATGGTCCACAGT |
| | GGCATGCACCT | TGATATCT | TTT |
| | SEQ ID NO: 50 | SEQ ID NO: 51 | SEQ ID NO: 52 |
| | | ACACCAGCCCTCAATACCACCCATATG | TTAGATCTAATAGTGATATAGCTTCTA |
| | | GTATCTGT | TGGTCCATAGTTT |
| | | SEQ ID NO: 53 | SEQ ID NO: 54 |
| | | | TCAGATCTAATAATGATATGGCTTCAA |
| | | | TGGTCCACAGTTT |
| | | | SEQ ID NO: 55 |
| 28 | GTGTCAAACAAAGGAGTAGATACTG | TTCCTTCCAGCTTGTTGACATAGTATAA | TAGGTTCCCCTTTTACATAAAGGTT |
| | SEQ ID NO: 71 | AGTGTTG | SEQ ID NO: 73 |
| | | SEQ ID NO: 72 | |
| 29 | TAGGAGCTATAGTGTCATGTTATGG | TGACACATAATCACAACCATTAGAAAAT | TCCTTCCAGCTTGTTTACATAGTAT |
| | SEQ ID NO: 74 | GTCTTTA | SEQ ID NO: 76 |
| | | SEQ ID NO: 75 | |
| 30 | TGGTTGTGATTATGTGTCAAACAAA | TAGATACTGTGTCAGTGGGCAACACTT | GCCTTCCAGCTTGTT |
| | SEQ ID NO: 77 | TATACTAT | SEQ ID NO: 79 |
| | | SEQ ID NO: 78 | |
| 31 | TCTAATGGTTGTGATTATGTGTCAA | CAAAGGAGTAGATACTGTATCAGTGGG | TCCTTCCAGCTTGTTTACATAGTAT |
| | SEQ ID NO: 80 | CAAC | SEQ ID NO: 76 |
| | | SEQ ID NO: 81 | |
| 32 | TCTAATGGTTGTGATTATGTGTCAA | CAAAGGAGTAGATACTGTATCAGTGGG | GCCTTCCAGCTTGTTGACATAGTAT |
| | SEQ ID NO: 80 | CAAC | SEQ ID NO: 82 |
| | | SEQ ID NO: 81 | |
| 33 | TGGTTGTGATTATGTGTCAAACAAA | TAGATACTGTGTCAGTGGGCAACACTT | TTTGCCTTCCAGCTTGTT |
| | SEQ ID NO: 77 | TATACTAT | SEQ ID NO: 83 |
| | | SEQ ID NO: 78 | |
| 34 | TCCCCTCTATGTACAACCAACACAA | TCCTGCATTATCACAATACCA | CTTTACATGTTTCAGCTTGTGGGA |
| | SEQ ID NO: 47 | SEQ ID NO: 84 | SEQ ID NO: 49 |
| 35 | TCCCCTCTATGTACAACCAACACAA | ATCCTGCATTATCACAATACCATCC | CTTTACATGTTTCAGCTTGTGGGA |
| | SEQ ID NO: 47 | SEQ ID NO: 85 | SEQ ID NO: 49 |
| 36 | TCCCCTCTATGTACAACCAACACAA | TCCTGCATTATCACAGTACCA | CTTTACATGTTTCAGCTTGTGGGA |
| | SEQ ID NO: 47 | SEQ ID NO: 86 | SEQ ID NO: 49 |
| 37 | TCCCCTCTATGTACAACCAACACAA | TACCATCCTCTATCAGTCCTTGTTA | CTTTACATGTTTCAGCTTGTGGGA |
| | SEQ ID NO: 47 | SEQ ID NO: 87 | SEQ ID NO: 49 |
| 38 | TCTAATGGTTGTGATTATGTGTCAA | CAAAGGAGTAGATACTGTATCAGTGGG | GCCTTCCAGCTTGTTGACATAGTAT |
| | SEQ ID NO: 88 | CAAC | SEQ ID NO: 90 |
| | | SEQ ID NO: 89 | |

Process Control (MS-2)

| | | | |
|---|---|---|---|
| 23 | GTTTCCGTCTTGCTCGTATC | CGCAAGTTCTTCAGCGAAAAGCAC | TTTCACCTCCAGTATGGAACC |
| | SEQ ID NO: 56 | SEQ ID NO: 57 | SEQ ID NO: 58 |
| 24 | CAATGCAACGTTCTCCAAC | TGCAGGATGCAGCGCCTTAC | TAACGGTTGCTTGTTCAGC |
| | SEQ ID NO: 59 | SEQ ID NO: 60 | SEQ ID NO: 61 |
| 25 | AATCTTCGTAAAACGTTCGTGTC | CACTTTTACCGTGGTGTCGATGTCAAA | CGAAGAGATTGTCAACAGGT |
| | | C | |
| | SEQ ID NO: 62 | SEQ ID NO: 63 | SEQ ID NO: 64 |
| 26 | GTCCGAGACCAATGTGC | CCGTTCCCTACAACGAGCCTAAATTCA | CAGGCAGCCCGATCTATT |
| | | TA | |
| | SEQ ID NO: 65 | SEQ ID NO: 66 | SEQ ID NO: 67 |
| 27 | ATCTTCGTAAAACGTTCGTGTCC | TTTGACATCGACACCACGGTAAAAGTG | GCGAAGAGATTGTCAACAGGTT |
| | SEQ ID NO: 68 | CG | SEQ ID NO: 70 |
| | | SEQ ID NO: 69 | |

A PCR primer set for amplifying an influenza A virus comprises at least one of the following sets of primer sequences: (1) SEQ ID NOS: 1 and 3; and (2) SEQ ID NOS: 4, 6, 7 and 8. A probe for binding to an amplicon(s) of an influenza A virus comprises at least one of the following probe sequences: SEQ ID NO: 2 and 5.

A PCR primer set for amplifying an influenza B virus comprises at least one of the following sets of primer sequences: (1) SEQ ID NO: 9 and 11; (2) SEQ ID NOS: 12, 14, 15 and 17; (3) SEQ ID NOS: 12, 17, 18, 19; (4) SEQ ID NOS: 12, 14, 17 and 19; (5) SEQ ID NOS: 12, 15, 17, 18; (6) SEQ ID NOS: 12, 15, 17, 20; (7) SEQ ID NOS: 15, 17, 18, 21; (8) SEQ ID NOS: 22, 24 and 26; (9) SEQ ID NOS: 12, 15 and 17; (10) SEQ ID NOS: 27 and 29; (11) SEQ ID NOS: 27 and 31: (12) SEQ ID NOS: 32, 34, 35 and 36; (13) SEQ ID NOS: 32, 34, 35 and 37; (14) SEQ ID NOS: 38 and 40 and (15) SEQ ID NOS: 41 and 43. A probe for binding to an amplicon(s) of an influenza B comprises at least one of the following probe sequences: SEQ ID NOS: 10, 13, 16, 23, 25, 28, 30, 33, 39 and 42.

A PCR primer set for amplifying an RSV comprises (1) SEQ ID NOS: 44 and 46; (2) SEQ ID NOS: 47 and 49; (3) SEQ ID NOS: 50, 52, 54 and 55; (4) SEQ ID NOS: 71 and 73; (5) SEQ ID NOS: 74 and 76; (6) SEQ ID NOS: 77 and 79; (7) SEQ ID NOS: 76 and 80; (8) SEQ ID NOS: 80 and 82; (9) SEQ ID NOS: 77 and 83 and (10) SEQ ID NOS: 88 and 90. A probe for binding to an amplicon(s) of an RSV comprises at least one of the following probe sequences: SEQ ID NO: 45, 48, 51, 53, 72, 75, 78, 81, 84, 85, 86, 87 and 89.

The probes can be molecular beacon probes, TagMan® probes, BHQ+ probes, and/or probes modified with locked nucleic acids.

The probes of the present invention are not limited to the modifications described herein. The probes of the present invention may be modified or unmodified.

Any set of primers can be used simultaneously in a multiplex reaction with one or more other primer sets, so that multiple amplicons are amplified simultaneously.

### Other Embodiments

Other embodiments will be evident to those of skill in the art. It should be understood that the foregoing detailed description is provided for clarity only and is merely exemplary. The spirit and scope of the present invention are not limited to the above examples, but are encompassed by the following claims. The contents of all references cited herein are incorporated by reference in their entireties.

What is claimed is:

1. A method of hybridizing one or more isolated nucleic acid sequences comprising contacting the one or more isolated nucleic acid sequences to a sample comprising the influenza A and/or influenza B and/or RSV virus(es) under conditions suitable for hybridization, wherein the isolated nucleic acid sequences are selected from the group of sequences consisting of: SEQ ID NOS: 1-55, 71-90 to an influenza A and/or influenza B and/or respiratory syncytial virus (RSV) virus(es).

2. The method of claim 1, wherein the influenza and/or RSV virus(es) is a genomic sequence, in a naturally occurring plasmid, in a naturally occurring transposable element, a template sequence or a sequence derived from an artificial construct.

3. The method of claim 1, further comprising:
(i) isolating the one or more hybridized target nucleic acids; and
(ii) sequencing the one or more hybridized target nucleic acids.

4. A primer set comprising at least one forward primer comprising a sequence selected from the group consisting of: SEQ ID NOS: 1, 4 and 7 (influenza A); 9, 12, 15, 19, 21, 22, 27, 32, 35, 38 and 41 (influenza B); and 44, 47, 50, 71, 74, 77, 80 and 88 (RSV), and at least one reverse primer comprising a sequence selected from the group consisting of: SEQ ID NOS: 3, 6 and 8 (influenza A); 11, 14, 17, 18, 20, 24, 26, 29, 31, 34, 36, 37, 40 and 43 (influenza B); and 46, 49, 52, 54, 55, 73, 76, 79, 82, 83 and 90 (RSV).

5. A method of producing a nucleic acid product, comprising contacting one or more isolated nucleic acid sequences selected from the group consisting of SEQ ID NOS: 1-55, 71-90 to a sample comprising an influenza A and/or influenza B and/or RSV virus(es) under conditions suitable for nucleic acid polymerization, wherein the nucleic acid product is optionally an amplicon produced using at least one forward primer comprising a sequence selected from the group consisting of: SEQ ID NOS: 1, 4 and 7 (influenza A); 9, 12, 15, 19, 21, 22, 27, 32, 35, 38 and 41 (influenza B); and 44, 47, 50, 71, 74, 77, 80 and 88 (RSV), and at least one reverse primer comprising a sequence selected from the group consisting of: SEQ ID NOS: 3, 6 and 8 (influenza A); 11, 14, 17, 18, 20, 24, 26, 29, 31, 34, 36, 37, 40 and 43 (influenza B); and 46, 49, 52, 54, 55, 73, 76, 79, 82, 83 and 90 (RSV).

6. A probe or set of probes that hybridizes to the nucleic acid product of claim 5, wherein the probe or set of probes optionally comprises one or more sequences selected from the group consisting of: SEQ ID NOS: 2, 5 (influenza A); 10, 13, 16, 23, 25, 28, 30, 33, 39 and 42 (influenza B); and 45, 48, 51, 53, 72, 75, 78, 81, 84, 85, 86, 87 and 89 (RSV), and wherein each probe is optionally labeled with a detectable label such as a fluorescent label, a chemiluminescent label, a quencher, a radioactive label, biotin and gold that is different from a detectable label associated with a different probe sequence.

7. A method for detecting influenza A, and/or influenza B, and/or RSV in a sample, comprising:
a) contacting the sample with at least one forward primer comprising a sequence selected from the group consisting of: SEQ ID NOS: 1, 4 and 7 (influenza A); 9, 12, 15, 19, 21, 22, 27, 32, 35, 38 and 41 (influenza B); and 44, 47, 50, 71, 74, 77, 80 and 88 (RSV), and at least one reverse primer comprising a sequence selected from the group consisting of: SEQ ID NOS: 3, 6 and 8 (influenza A); 11, 14, 17, 18, 20, 24, 26, 29, 31, 34, 36, 37, 40 and 43 (influenza B); and 46, 49, 52, 54, 55, 73, 76, 79, 82, 83 and 90 (RSV), under conditions such that nucleic acid amplification occurs to yield an amplicon; and
b) contacting the amplicon with one or more probes comprising one or more sequences selected from the group consisting of: SEQ ID NOS: 2, 5 (influenza A); 10, 13, 16, 23, 25, 28, 30, 33, 39 and 42 (influenza B); and 45, 48, 51, 53, 72, 75, 78, 81, 84, 85, 86, 87 and 89 (RSV), under conditions such that hybridization of the probe to the amplicon occurs;
wherein hybridization of the probe is indicative of influenza A, influenza B and/or RSV in the sample.

8. The method of claim 7, wherein the sample is selected from the group consisting of: saliva, fluids collected from the ear, eye, mouth, and respiratory airways, sputum, tears, oropharyngeal swabs, nasopharyngeal swabs, nasal swabs, throat swabs, nasopharyngeal aspirates, bronchoalveolar lavage fluid, skin swabs, nasal aspirates, nasal wash, and fluids and cells obtained by the perfusion of tissues of both human and animal origin.

9. The method of claim 8, wherein the sample is derived from a human or non-human or an inanimate object or environmental surface.

10. A kit for detecting influenza A, and/or influenza B, and/or RSV virus in a sample, comprising one or more probes comprising a sequence selected from the group consisting of: SEQ ID NOS: 2, 5 (influenza A); 10, 13, 16, 23, 25, 28, 30, 33, 39 and 42 (influenza B); and 45, 48, 51, 53, 72, 75, 78, 81, 84, 85, 86, 87 and 89 (RSV), wherein the kit optionally further comprises:
a) at least one forward primer or primer pair comprising the sequence selected from the group consisting of: SEQ ID NOS: 1, 4 and 7 (influenza A); 9, 12, 15, 19, 21, 22, 27, 32, 35, 38 and 41 (influenza B); and 44, 47, 50, 71, 74, 77, 80 and 88 (RSV); and
b) at least one reverse primer or primer pair comprising the sequence selected from the group consisting of: SEQ ID NOS: 3, 6 and 8 (influenza A); 11, 14, 17, 18, 20, 24, 26, 29, 31, 34, 36, 37, 40 and 43 (influenza B); and 46, 49, 52, 54, 55, 73, 76, 79, 82, 83 and 90 (RSV); and/or
c) reagents for sequencing influenza A and/or influenza B and/or RSV virus in the sample; and/or.
d) a process control optionally further comprising a process control probe, process control forward primer and process control reverse primer comprising the sequence selected from the group consisting of: SEQ ID NOS: 56, 59, 62, 65 and 68 (process control forward primers); 58, 61, 64, 67 and 70 (process control reverse primers); and 57, 60, 63, 66 and 69 (process control probes).

11. The kit of claim 10, wherein the one or more probes are labeled with different detectable labels and wherein the one or more probe sequences are optionally labeled with the same detectable label.

12. A method of diagnosing a condition, syndrome or disease in a human associated with an influenza A and/or influenza B and/or RSV virus comprising:
a) contacting a sample with at least one forward and reverse primer set comprising a sequence selected from the group consisting of: (1) SEQ ID NOS: 1 and 3; (2) SEQ ID NOS: 4, 6, 7 and 8; (3) SEQ ID NO: 9 and 11; (4) SEQ ID NOS: 12, 14, 15 and 17; (5) SEQ ID NOS: 12, 17, 18, 19; (6) SEQ ID NOS: 12, 14, 17 and 19; (7) SEQ ID NOS: 12, 15, 17, 18; (8) SEQ ID NOS: 12, 15, 17, 20; (9) SEQ ID NOS: 15, 17, 18, 21; (10) SEQ ID NOS: 22, 24 and 26; (11) SEQ ID NOS: 12, 15 and 17; (12) SEQ ID NOS: 27 and 29; (13) SEQ ID NOS: 27 and 31: (14) SEQ ID NOS: 32, 34, 35 and 36; (15) SEQ ID NOS: 32, 34, 35 and 37; (16) SEQ ID NOS: 38 and 40; (17) SEQ ID NOS: 41 and 43; (18) SEQ ID NOS: 44 and 46; (19) SEQ ID NOS: 47 and 49; (20) SEQ ID NOS: 50, 52, 54 and 55; (21) SEQ ID NOS: 71 and 73; (22) SEQ ID NOS: 74 and 76; (23) SEQ ID NOS: 77 and 79; (24) SEQ ID NOS: 76 and 80; (25) SEQ ID NOS: 80 and 82; (26) SEQ ID NOS: 77 and 83 and (27) SEQ ID NOS: 88 and 90.
b) conducting an amplification reaction, thereby producing an amplicon; and
c) detecting the amplicon using one or more probes comprising a sequence selected from the group consisting of: SEQ ID NOS: 2, 5, 10, 13, 16, 23, 25, 28, 30, 33, 39, 42, 45, 48, 51, 53, 72, 75, 78, 81, 84, 85, 86, 87 and 89;
wherein the detection of an amplicon is indicative of the presence of an influenza A and/or influenza B and/or RSV virus in the sample, wherein optionally the sample is selected from the group consisting of: saliva, fluids collected from the ear, eye, mouth, and respiratory airways, sputum, tears, oropharyngeal swabs, nasopharyngeal swabs, nasal swabs, throat swabs, nasopharyngeal aspirates, bronchoalveolar lavage fluid, skin swabs, nasal aspirates, nasal wash, and fluids and cells obtained by the perfusion of tissues of both human and animal origin.

13. The method of claim 12, wherein the condition, syndrome or disease in a human associated with an influenza A and/or influenza B and/or RSV virus is selected from the group consisting of: asthma, middle ear infection, bronchiolitis, fever, chills, anorexia, headache, myalgia, weakness, sneezing, rhinitis, sore throat, cough, nausea, vomiting, pneumonia, death, afebrile respiratory illnesses, myositis, rhabdomyolysis, myalgias, central nervous system disease (CNS) including encephalitis, transverse myelitis, aseptic meningitis, and Guillain-Barré syndrome (GBS).

14. A process control primer set comprising at least one forward primer comprising a sequence selected from the group consisting of: SEQ ID NOS: 56, 59, 62, 65 and 68; and
at least one reverse primer comprising a sequence selected from the group consisting of: SEQ ID NOS: 58, 61, 64, 67 and 70.

15. A process control probe comprising a probe comprising a sequence selected from the group consisting of: SEQ ID NOS: 57, 60, 63, 66 and 69, wherein the probe is optionally labeled with a detectable label selected from the group consisting of: a fluorescent label, a chemiluminescent label, a quencher, a radioactive label, biotin and gold.
